(19) United States
(12) Patent Application Publication
(10) Pub. No.: US 2004/0117125 A1
(43) Pub. Date: Jun. 17, 2004

(54) Drug discovery method and apparatus

(76) Inventors: Hao Chen, Columbia, MD (US); David M. Manyak, Ellicott City, MD (US); Peter S. Carlson, Chevy Chase, MD (US); Fong Liu Wang, Potomac, MD (US); Ming Liu, Rockville, MD (US)

Correspondence Address: Finnegan, Henderson, Farabow,, Garrett & Dunner, L.L.P., 1300 I Street, N.W., Washington, DC, 20005-3315, US

(21) Appl. No.: 10/669,705

(22) Filed: Sep. 25, 2003

Related U.S. Application Data
(63) Continuation-in-part of application No. 10/394,586, filed on Mar. 24, 2003, now abandoned.
(63) Continuation-in-part of application No. 10/105,407, filed on Mar. 26, 2002.
Application No. 10/105,407 is a continuation-in-part of application No. 09/558,232, filed on Apr. 26, 2000.
(60) Provisional application No. 60/366,576, filed on Mar. 25, 2002.
(60) Provisional application No. 60/130,992, filed on Apr. 26, 1999.

(51) Int. Cl. G06F 19/00; G01N 33/48; G01N 33/50; G01N 31/00

(52) U.S. Cl. 702/19; 702/22

(57) ABSTRACT

Methods and systems for drug discovery and development are disclosed. Methods and system consistent with the present invention discover drugs. One or more databases comprising chemical and biological interaction data and one or more computer-based data analysis programs may be used to identify compounds that have desired activity at two or more molecular targets that are associated with a disease state for which the drug discovery and development are directed. In addition, one or more databases comprising chemical and biological interaction data and one or more computer-based data analysis programs may be used to identify compounds that (a) have desired activity at one or more molecular targets that are associated with a disease state for which the drug discovery and development are directed and (b) do not have activity or have substantially reduced activity that is undesired at one or more molecular targets that are associated with possible side effects, toxicity, adverse ADME properties, or other properties not intended to be manifested by compounds being developed to treat the disease state associated with the drug discovery.

### RELATED APPLICATIONS

This application is a continuation-in-part of U.S. application Ser. No. 10/394,586, filed Mar. 24, 2003, which claims the benefit of U.S. Provisional Application No. 60/366,576, filed Mar. 25, 2002. This application is also a continuation-in-part of U.S. application Ser. No. 10/105,407, filed Mar. 26, 2002, which is a continuation-in-part of U.S. application Ser. No. 09/558,232, filed Apr. 26, 2000, which claims the benefit of U.S. Provisional Application No. 60/130,992, filed Apr. 26, 1999.

### TECHNICAL FIELD

The field of the invention relates to methods and systems for drug discovery and development.

### BACKGROUND OF THE INVENTION

The traditional paradigm for drug discovery and development has been basically a linear process. During the early stages of the drug discovery process, large compound libraries, numbering hundreds of thousands to millions of chemical compounds (synthetic, small organic molecules or natural products, for example) are screened or tested for biological activity at any one of hundreds of molecular targets in order to find potential new drugs, or lead compounds. The active compounds, or hits, from this initial screening process are then tested sequentially through a series of other in vitro and in vivo tests to further characterize the active compounds. A progressively smaller number of the presumptive “best” compounds at each stage are selected for testing at the next stage, eventually leading to one or at most a few drug candidates (for those “successful” discovery programs) being selected to proceed to Investigational New Drug (IND) status and be tested in human clinical trials. If, at any stage along the linear sequence of tests and decision points, a hit, lead compound, or drug candidate fails to meet the standards for continued development as a drug, the process of discovery and development must start over again. Unfortunately, under the traditional paradigm, the failure rate is high—more than 90% of drug candidates that reach IND status fail to gain marketing approval by the Food and Drug Administration (FDA). About one-half of these failures are due to undesirable or adverse side effects and the other half to insufficient efficacy.

The pharmaceutical industry has directed its past drug development efforts at only about 500 pharmacological targets, which are generally proteins such as receptors or enzymes associated with disease states. As a result of efforts to sequence the human genome, it now appears that there may be a total of 10,000 pharmaceutically relevant protein targets. This represents a 20-fold increase in the number of drug targets that may be addressable in the next decade. At the same time, advances in the automation of chemical synthesis, commonly known as combinatorial chemistry, have led to substantial increases in the size of chemical libraries available to the drug industry to screen against pharmacological targets for drug discovery. As a result, compound libraries at major drug companies are now some 10-fold larger that they were just three-to-five years ago, numbering well over 1,000,000 chemicals at many companies.

Although new drug discovery technologies have produced an explosion in the number of compounds emerging from the initial discovery phase, this has not translated into a proportional increase in new and safer drugs reaching the market. Genomics, combinatorial chemistry and high-throughput screening have produced more drug targets and more compounds to screen in a more rapid format, but the end result remains largely unchanged. Lead compound attrition has now become the primary problem for the industry. A majority of the small organic molecules that emerge from drug discovery with confirmed biological activity against a macromolecular drug target will fail in some subsequent stage of the development process. Often such problems do not become evident until the lead compound has reached Phase II or Phase III human clinical trials. This means that the drug development company has wasted substantial time, money and effort. There is a need to understand what causes failure in the late stages of drug development and to correct the discovery process at the early stages to minimize those late-stage failures.

Drug Efficacy and Safety—There are many pharmaceutical companies, large and small, domestic and international. Yet, the primary model of current drug discovery and the infrastructure of the industry are essentially identical. Conventional approaches to drug discovery focus on chemical intervention at a single biochemical target or mechanism. Based on this concept, the aims of drug discovery and development are to find and to produce small molecules that are highly specific with respect to one specific macromolecule, with the intent of potently intervening, interrupting and modulating the biochemical or biological function of a single biological target. The hope of the pharmaceutical industry is that such potent “interruption or modulation” will produce some beneficial effects ameliorating certain conditions associated with disease progression.

In contrast to the drug discovery industry, medical practitioners take a different approach. Clinicians often resort to multiple drug cocktails for disease treatments. One of the well-know examples of multiple drug combinations is in the treatment of AIDS by employing cocktails of reverse transcriptase inhibitors and protease inhibitors. Another example is in the treatment of bacterial infections employing lactamase inhibitors (e.g., clavulanate) with cell wall synthesis inhibitors, and yet another example is in hypertension management employing ACE inhibitors along with diuretic drugs. Drug manufacturers have also adopted this approach and have developed similar products for management of many chronic diseases. For example, CombiVent, a medication for asthma, is a combination of a muscarinic (M₃) antagonist and a beta adrenoceptor-blocker (beta-2); Claritin-D, an over-the-counter (OTC) allergy medication, is a combination of Loratadine (antihistamine) and pseudoephedrine. In fact, in recent years, examples of drug combinations or multi-drug regimens have become commonplace in medical practice.

Developing multiple drug ingredients for a single medication multiplies the cost of discovery and lengthens the development process, which ultimately increases the cost and quality of health care. One can readily observe this fact on the store shelf where the cost of Claritin-D (Claritin plus Sudafed) is significantly higher than that of each drug ingredient alone.

Monetary concerns aside, the most serious concern about poly-drug regimens is safety. In a recent report, Urs Meyer pointed out that drug interactions may cause 100,000 deaths per year in the U.S. This figure makes adverse drug interactions somewhere between the fourth and sixth leading cause of death among hospitalized patients.

In short, clinical experiences have indicated that in order to effectively treat many disease conditions, acute or chronic, clinicians must simultaneously address multiple biological events. Treatment of AIDS requires concurrent inhibition of protease activity and reverse transcriptase activity. Hypertension management at best requires the management of both vasoconstriction (vascular resistance, ACE inhibitors) and ion transport and balance (volume reduction, diuretics). Such phenomena are a demonstration of the redundancies inherent in the physiological controls characteristic of resilient, robust, stable and highly complicated biological systems. However, when the functioning of such systems needs to be corrected, modulated or controlled under disease conditions, a multitude of biological events must be simultaneously considered, addressed, stimulated and/or attenuated. Potentially dangerous, multiple drug combinations or regimens are, so far, often the only means of accomplishing the multiple physiological effects required for effective disease control.

Ideally, to develop safe and efficacious drugs, the requirement is to find a single chemical entity with an activity profile addressing more than one biochemical or biological pathway and/or more than one physiological mechanism. In contrast, at present, the drug discovery and development process is ill equipped to meet these demands. The industry-wide high throughput-screening paradigm normally generates about 0.1% hit rates during a screening “campaign” (a word coined in the industry to illustrate the scale of a project, or the level of industrial madness) of a compound library against a single biological target. With the same paradigm, looking for compounds that are concurrently active against two targets, the hit rate will typically be only a small fraction of the 0.1% hit rate per single target, or statistically a probability of less than one in a million compounds (0.1% for target #1 times 0.1% for target #2). By industry standards, a successful primary (initial) screening run is benchmarked as finding hits in more than five chemical structural classes (with more than a single hit in each class), meaning the one in a million probability for hitting both targets yields a need for at least 10 hits (5 classes; 2 hits/class) from 10 million compounds tested. Such a massive scale of screening run is impractical, and in fact the costs to implement this approach would be enormous.

Unintended Biological Effects and Other Contributing Factors—Because of the lack of an ability or technique to simultaneously handle multiple biological concerns and issues, the industry-wide process of drug discovery and development is now a primarily linear and stepwise process. By testing in sequential events, from in vitro to in vivo, from test tube to live primates, from IND to post market monitoring, compounds, hits, leads and candidates are triaged for desired vs. undesired properties physically, chemically, biochemically and then clinically. The high attrition rate at each of these sequential steps creates a process that is arduous, lengthy, and plagued with failures. With each progressive step in the drug discovery process, the costs escalate. The cost of high throughput screening on average is about $0.50 to $1.00/compound; the cost of animal testing for safety of a single lead candidate is in the range of hundreds of thousands of dollars; the cost of clinical trials for one candidate is on the scale of multiples of millions of dollars. Therefore, it is a requirement for the pharmaceutical industry to accurately eliminate any lead compounds that display any potential uninitendeu biological effects early in the discovery process when the costs associated with testing and triage for that compound are still minimal.

One way to avoid these hidden, undesired and unintended biological effects associated with lead compounds, which contribute to expensive failures in drug development, is to optimize the pharmacological properties of the compounds early in the development process when the cost is relatively low. The pharmacological properties may include the compound's potency of activity with respect to the intended target or targets, as well as its lack of activities with respect to targets that may be contributing deleterious side effects. However, when compounds are found to be “reactive” with more than one biological target they are often inherently promiscuous within the general pharmacological target class. Hence it is even more important to uncover and eliminate those compounds that display undesired promiscuity early in the drug discovery process.

In summary, drug candidates fail to become marketed pharmaceuticals primarily because of two issues, efficacy and unintended effects. It is the overall biological activity profile (however measured) of a chemical that ultimately decides the fate of whether this chemical is a drug candidate and becomes a marketed drug or not. In order to avoid downstream failures (e.g., in Phase II or III clinical trials, for instance), the discovery-development paradigm needs to take multiple issues, i.e., (i) selection of one or more biological targets covering multiple biochemical and/or physiological mechanisms of actions and (ii) optimal pharmacological activity profiles across multiple potential side effect, toxicology, and/or pharmacokinetics-related targets, into consideration early in the drug discovery process. Currently, the industry wide paradigms and available technologies are not capable of adequately meeting this need.

### SUMMARY OF THE INVENTION

Systems and methods consistent with the present invention provide utilities of a knowledge base of molecular interactions between a wide range of pharmaceutically relevant molecular targets and broad set of information rich chemicals determined empirically in the laboratory to serve as a dataset for modeling molecular recognition (the reactivity selectivity mapping database, or RSMDB).

Systems and methods consistent with the present invention also include information in the knowledge base or database about the molecular targets (bioinformatic annotations), chemical compounds (chemoinformatic annotations), and codes describing the structural features of both the targets and chemicals (descriptors), all of which are used in describing the patterns of molecular recognition;

Systems and methods consistent with the present invention also use the database structure and related software to organize and analyze the target and compound interaction data, annotations, and descriptors and provide output in forms, including predictive algorithms, that describe key aspects of molecular recognition.

Systems and methods consistent with the present invention also indicate that the database provides arrays of information concurrently for one or more biological targets representing biological effects intended to direct medication development.

More particularly, in systems and methods consistent with the present invention, one or more databases comprising chemical and biological interaction data and one or more computer-based data analysis programs may be used to identify compounds that have desired activity at two or more molecular targets that are associated with a disease state for which the drug discovery and development are directed.

Also in systems and methods consistent with the present invention, one or more databases comprising chemical and biological interaction data and one or more computer-based data analysis programs may be used to identify compounds that (a) have desired activity at one or more molecular targets that are associated with a disease state for which the drug discovery and development are directed and (b) do not have activity or have substantially reduced activity that is undesired at one or more molecular targets that are associated with possible side effects, toxicity, adverse ADME properties, or other properties not intended to be manifested by compounds being developed to treat the disease state associated with the drug discovery.

In yet other systems and methods consistent with the present invention, two or more molecular targets related to a cause or mechanism of a disease, disease process or medical condition are selected. A dataset comprising results of tests of interactions between each of the selected targets and a multiplicity of chemical compounds may also be accessed, wherein the chemical compounds may be described by descriptors related to features of the compounds. Criteria for selecting those chemical compounds that demonstrate activity in the tests of interactions between the targets and compounds are then established, for each of the selected molecular targets. Those compounds are selected based on the established criteria. The system thereafter assembles sets of descriptors that are identified with those compounds comprising the set of selected active compounds, for each of the selected molecular targets; identifies, from the sets of assembled descriptors for each selected molecular target, those descriptors that are found in common for each combination of two or more of the selected molecular targets; and identifies, using the identified in common descriptors, chemical compounds useful for drug discovery purposes related to a disease, disease process, or medical conditions to which the selected molecular targets are related.

### BRIEF DESCRIPTION OF THE DRAWINGS

The accompanying drawings, which are incorporated in and constitute a part of this specification, illustrate one embodiment of the invention and, together with the description, serve to explain the principles of the invention.

FIG. 1 is a diagram of a process for in silico screening consistent with the present invention;

FIG. 2 is a diagram of a process for in silico screening using pharmacoinformatics consistent with the present invention;

FIG. 3 shows an equation defining a pharmacological profile consistent with the present invention;

FIG. 4 depicts a tree hierarchy of compounds consistent with the present invention;

FIG. 5 depicts a partial D1 tree image consistent with the present invention;

FIG. 6 shows an exemplary 2D bond distance descriptors set consistent with the present invention;

FIG. 7 shows a diagram of exemplary receptor/transporter systems consistent with the present invention;

FIG. 8 shows another diagram of exemplary receptor/transporter systems consistent with the present invention;

FIG. 9 represents a typical case of using recursive partitioning to identify chemical descriptors consistent with the present invention;

FIG. 10 shows a partial dataset representing the optimized probability of finding compounds modulating activities at multiple biological targets consistent with the present invention;

FIG. 11 shows an exemplary demonstration of me interactions of a drug can diate with molecular targets consistent with the present invention;

FIG. 12 shows an exemplary activity profile of 406 compounds screened against 7 GPCR targets consistent with the present invention;

FIG. 13 depicts reactivity profiles of 9 compounds that showed nearly specific activity more reactivity with D1 than other compounds of the same array;

FIG. 14 shows an initial data set obtained from testing a panel of 600 compounds against dopamine D1 (X) and adenosine 2A (Y) activity;

FIG. 15 shows an activity profile of a lead compound demonstrating concurrent activity with D1 and Adenosine A2a;

FIG. 16 shows a discovery/development strategy consistent with the present invention;

FIG. 17 shows the interrelationship between a pharmacoinformatics database and in silico screening methods consistent with the present invention;

FIG. 18 shows a list of chemical compound types that may be included in a pharmacoinformatics database consistent with the present invention;

FIG. 19 shows a list of molecular target types that may be included in a pharmacoinformatics database consistent with the present invention;

FIG. 20 shows a timeline for drug discover and development consistent with the present invention; and

FIG. 21 shows an example of potential time and cost savings achievable using methods consistent with the present invention.

### DETAILED DESCRIPTION OF THE INVENTION

Reference will now be made in detail to exemplary embodiments of the present invention, examples of which are illustrated in the accompanying drawings. While the description includes exemplary embodiments, other embodiments are possible, and changes may be made to the embodiments described without departing from the spirit and scope of the invention. The following detailed description does not limit the invention. Instead, the scope of the invention is defined by the appended claims and their equivalents.

The present invention discloses a novel approach to drug discovery and developimtenit using a database that encompasses drug discovery information presented as:

(1) Chemoinformatic information, including chemical structures and related physical and/or chemical and/or physicochemical descriptors that are sufficient in describing a molecule, be it man-made or found in nature.

(2) Bioinformatic information, including the name and structural information describing a macromolecule, which could be a protein representing a membrane receptor, a nuclear receptor, an enzyme, an ion channel or a conductance regulator, a compound transporter or the like. The macromolecule may also be specified as segments of polymeric nucleic acids (DNA or RNA) with specific or given sequences. The bioinformatic information may also be represented by specific nucleic acid or amino acid (peptide) sequences or other descriptors of that macromolecule that identifies the nature of the macromolecule.

(3) Information comprising, describing and detailing the various interactions between the “stored” chemoinformatic and bioinformatic information; the interactions being derived, measured or observed using any physical, chemical, or biological means, and recorded and described either quantitatively or qualitatively. The recorded interactions may be numerical or descriptive in nature.

More information on such a database may be found in U.S. application Ser. No. 09/558,232, filed on Apr. 26, 2000, which is incorporated by reference. Using the described database, the present invention is a method of detecting, identifying and/or designing small organic molecules displaying a defined profile of biological activities. That is, the database can be examined and queried using data interrogation tools that employ a wide assortment of data-correlation methodologies based on a variety of algorithms. The desired pattern and design of the interaction profiles of the small organic molecules may be comprised of a multitude of biological activities with macromolecules, selected therapeutic concerns, and related physiological phenomena. This pattern and design may include identification of a chemical entity that is reactive or is not reactive with a defined assortment of related macromolecular biological targets or is reactive or is not reactive with an array of related or unrelated biochemical mechanisms. The present invention facilitates an increase in productivity of drug discovery activities and represents a novel methodology as embodied and enabled in the examples and illustrations.

One chailenge for the pharmaceutical and biotechnology industries is to correlate their past successes or failures in drug development and commercialization, as measured both in terms of in vivo activity of chemical compounds and in vitro reactivity of chemical compounds with a broad range of molecular targets, with the chemical structures of these molecules and then to use that knowledge base to create new molecules that do not possess any causes of failure. There is a substantial need to determine with increased efficiency the eventual success or failure of candidate drug molecules and front-load the drug discovery process with predictive information and tools that will directly lead to new, innovative drugs.

New databases have emerged in the life sciences in recent years to manage and interpret new sources of genomic and chemical data. Databases of genetic sequence, proteomic, and functional genomic information are well-established and have created numerous successful businesses (an area known as “bioinformatics”). Similarly, chemical structure databases (“chemoinformatics”) are well-established in the drug industry. This series of databases describes the biological components (e.g., DNA, proteins, and small molecule effectors), and the interactions between these components, involved in basic life processes, as well as in drug discovery. However, the missing link in this series is “pharmacoinformatics,” that is, the molecular recognition and interactions between proteins, such as receptors or enzymes, and small molecules or drugs. This is the critical interface at which informatics can be applied to more accurately identify or predictably design new drug candidates that have the greatest probability of successfully reaching the market as a new pharmaceutical. Moreover, using computer-based (“in silico”) screening rather than brute-force high throughput screening based on in vitro assays promises to dramatically reduce the cost of the entire drug discovery process, as well as making it more accurate.

The ability to create and deploy pharmacoinformatics strategies to solve the difficult problems facing pharmaceutical R&D first and foremost requires a comprehensive, highly informative dataset that can be used to “train” the data mining software, generate the predictive algorithms, and enable in silico screening approaches. Such a dataset requires an extremely broad array of molecular-target-based screening assays and a highly informative, rationally selected chemical library, plus implementation of multiplexed screening strategies and production of a high quality dataset. Previously, that dataset has not existed in the drug industry. An example of such a dataset may be found in U.S. application Ser. No. 09/558,232, filed on Apr. 26, 2000.

Pharmacoinformatics is directed toward the interface between biological target information (“bioinformatics”) and chemical compound information (“chemoinformatics”). This informatics integration is designed to model or predict the key physical interactions between biological targets and chemical compounds, an event called molecular recognition. The process of molecular recognition, or binding between a chemical and target, is very specific, much like a key fits a specific lock. Often the targets are receptors, transporters, ion channels, etc., or enzymes that mediate key events in cells and are naturally modulated by native chemicals (called “ligands” for receptors or “substrates” for enzymes) in the body designed by nature to control cellular functions. New chemicals, or drugs, that intervene in this interaction between targets and native ligands or substrates can either enhance (“agonists”) or block (“antagonists” or “inhibitors”) the natural process.

When designing a drug discovery program, a molecular target can be assembled into a screening assay, and a series of chemicals tested empirically to determine which chemicals demonstrate molecular recognition by virtue of their binding interaction with the target. Such binding interactions can lead to functional biological activity. Screening assays can also be designed to directly test for functional activity as a measurement of interaction. Many different chemicals may interact with one target. At the same time, many targets have sufficiently similar features that any one chemical may interact with numerous targets. If a chemical that interacts with the intended molecular target also interacts with a target that mediates an undesirable effect, or potential cause of a side effect, it would be less attractive as a drug candidate than one that interacts only with the intended target. Because of the large number of similar targets (such as receptors in the central nervous system, for example) that can be either therapeutic targets or side effect targets, depending on the desired use of the drug candidate, determining the relative molecular recognition for selected compounds across such a range of targets can be a daunting task. In the linear practice of drug discovery today, that means repetitive screening of drug candidates across numerous targets—a process called “selectivity screening” or “profiling.”

According to one embodiment of the present invention, a pharmacoinformatics technology platform creates a knowledge base of individual interactions between molecular targets and chemicals and uses that information, together with software and data mining tools, to derive patterns of molecular recognition that can be predictive for Key aspects of drug discovery and development. For example, pharmacoinformatics can be used to predict which features of chemicals (substructural components or “descriptors”) are associated with molecular recognition with the intended target but are not recognized by a range of other targets that may mediate certain side effects. Or, for example, pharmacoinformatics can be used to predict which chemical features or descriptors in common are associated with molecular recognition with two or more intended targets that are both (or multiply) involved in a specific disease or condition to which the discovery program is directed. The prediction can be done rapidly on a computer, turning the discovery process into an efficient parallel process rather than a costly, time-consuming linear process based on sequential laboratory screening.

According to one embodiment of this invention, the pharmacoinformatics technology consists of the following:

1. A knowledge base of molecular interactions between a wide range of pharmaceutically relevant molecular targets and broad set of information rich chemicals determined empirically in the laboratory to serve as a dataset for modeling molecular recognition (the Receptor Selectivity Mapping Database or Reactivity:Selectivity Mapping Database, or RSMDB);

2. Information about the molecular targets (bioinformatic annotations) and chemical compounds (chemoinformatic annotations) and codes describing the structural features of both the targets and chemicals (descriptors), all of which are used in describing the patterns of molecular recognition; and

3. Database structures and software used to organize and analyze the target and compound interaction data, annotations, and descriptors and provide output in forms including predictive algorithms that describe key aspects of molecular recognition.

RSMDB Content Databases

An RSMDB dataset is created for the pharmacoinformatics platform by using in vitro screening assays to establish a matrix of information of measured molecular interactions between a set of information-rich chemical compounds and a wide panel of pharmaceutically relevant molecular targets. Features of this RSMDB are (1) the choice of chemical compounds and (2) molecular targets, and that the screening or molecular interaction dataset is (3) full-rarn- and I high-density, (4) quantitative, and (5) internally consistent.

Chemical Compounds. The compound library for the RSMDB consists of the following major categories:

1. marketed pharmaceuticals (U.S. and foreign);

2. over-the-counter (OTC) medications and ingredients;

3. marketed agricultural chemicals/veterinary medicines;

4. failed or discontinued drug candidates; drugs withdrawn from the market;

5. drug candidates in clinical trials;

6. pharmacological reference agents and bioactive natural products; and

7. structurally diverse chemicals without known biological activity.

The selection of compounds for the RSMDB dataset is biased toward those having demonstrated biological activity, combined secondarily with a set of compounds that broadens the diversity of chemical structural features represented in the database. This bioactive-biased set yields important advantages to the database for statistical modeling purposes. Another important result of this compound selection is that the RSMDB contains screening or interaction data for most marketed pharmaceuticals and other compounds with known acceptable safety profiles against a broad set of pharmaceutically relevant targets, which dataset, in another embodiment of the present invention, can be mined directly to search for new therapeutic applications of existing drugs and other low-risk compounds.

Molecular Targets. The array of molecular targets for the RSMDB includes both receptors (including related targets such as ion channels, transporters or re-uptake sites, etc.) and enzymes and consists of the following major categories:

1. in vitro pharmacology: primary therapeutic or disease-related targets;

2. in vitro pharmacology: targets associated with drug side effects or off-target effects;

3. in vitro toxicology: toxic effects of compounds; and

4. in vitro pharmacokinetics: drug absorption, distribution, metabolism, and excretion.

Nearly all major categories of receptor classes and most receptor subtypes in these classes are or can be included in the RSMDB. These receptors, especially a group called the G-Protein Coupled Receptors (GPCRs) and/or seven transmembrane receptors (7TMs), represent the primary therapeutic targets for more than 50% of all current drug sales. Furthermore, many of these same receptor classes mediate key unwanted side effects of drugs. Target classes for drug action can be generally classified as follows:

1. GPCRs/7TMs

2. Nuclear hormone receptors

3. Ion channels

4. Transporters or re-uptake sites

5. Enzymes, including proteases, kinases, metabolic enzymes, etc.

The RSMDB may contain representatives of all five types of targets. A number of enzyme targets that mediate toxicity (e.g., caspases) or pharmacokinetics (e.g., cytochrome P450s) can also be included in the RSMDB dataset. In one embodiment of the present invention, the RSMDB dataset contains more than 90 different targets, about two-thirds of which are GPCRs/7TMs. Fewer or more targets included within the RSMDB dataset is also within the scope of the current invention, provided however that a multiplicity of targets is required for the invention. Considering that the entire number of targets addressed in the history of the drug industry, until recently, was only 500, the RSMDB dataset can represent a substantial cross-sectional map of existing pharmaceutical space, in terms of molecular recognition. Note that RSMDB is a full-rank database in terms of protein-ligand binding, which means that binding data of each compound is tested against each protein available regardless of whether it is inactive or active.

Chemoinformatic/Bioinformatic Annotations and Descriptors

The RSMDB dataset may be organized into an Oracle database with a table structure to facilitate input/organization, search/retrieval, analysis/mining, and visualization/output of the information. Oracle tables can hold the screening dataset as well as chemoinformatic annotations (such as chemical structure in digital format such as sd files or mol files, molecular weight, solubitiy, IUPAC name, etc.) on the RSMDB chemicals and bioinformatic annotations (such as amino acid sequence, gene accession number reference, target family/classification, etc.) on the RSMDB targets. Sets of descriptors, which are digitally-formatted codes that describe the substructural features of chemical compounds and molecular targets, as well as other information, can further be built into the pharmacoinformatics platform. The chemical descriptors allow the dataset to be expanded into a far greater variety of chemical compounds than just the RSMDB compounds themselves. They are also critical for in silico screening.

Data Mining Tools and Predictive Algorithms

Data mining approaches for drug discovery and development can be based on use of the RSMDB content database as a knowledge base or “training set,” Oracle or other table structures, and application software with a range of statistical methods. One such statistical approach is recursive partitioning in which descriptor datasets are sequentially queried for the probability of, for example, specific descriptors from among a group of descriptor types being correlated with molecular recognition at a single target in the RSMDB. Each sequential query gives a yes-no branching that is continued until the branches of the tree terminate with the highest probability descriptor(s) for molecular recognition. In its simplest form, this descriptor set can then be used as the basis for in silico screening at a single target. In its more complex form, in one embodiment of the present invention, the recursive partitioning can be performed for multiple targets to derive those descriptors that correlate with positive activity for the intended molecular target but lack of activity at similar targets that might cause a side effect or adverse toxicological or pharmacokinetic effect. In another embodiment of the present invention, the recursive partitioning can be performed for multiple targets to derive those descriptors that correlate with positive activity for two or more intended molecular targets that are associated with a specific disease or condition that is the subject of the drug discovery program (therapeutic targets). In yet another embodiment of the present invention, the recursive partitioning can be performed for multiple targets to derive those descriptors that correlate with positive activity for two or more intended therapeutic targets but lack of activity at similar targets that might cause a side effect or adverse toxicological or pharmacokinetic effect. Those predictive algorithms can then be used for in silico screening. Other statistical methods can be used, adapted, and/or developed for the pharmacoinformatics platform.

In Silico Screening Approaches for Drug Discovery

The most direct initial application of pharmacoinformatics for in silico screening approaches is for drug discovery at a selected molecular target. A discovery target that is in the RSMDB or related by descriptors and the RSMDB dataset can be analyzed by, e.g., recursive partitioning to derive an algorithm defining which chemical descriptors are predictive of molecular recognition or desired activity at that discovery target. A large chemical library for which all the compounds are also digitally represented and broken down into descriptors is then scanned for the presence of the desired descriptor(s). This generates a “virtual” compound library of much smaller size. Those compounds are selected from the libraries, acquired, and physically screened at the discovery target using the in vitro assay to confirm the predicted activity.

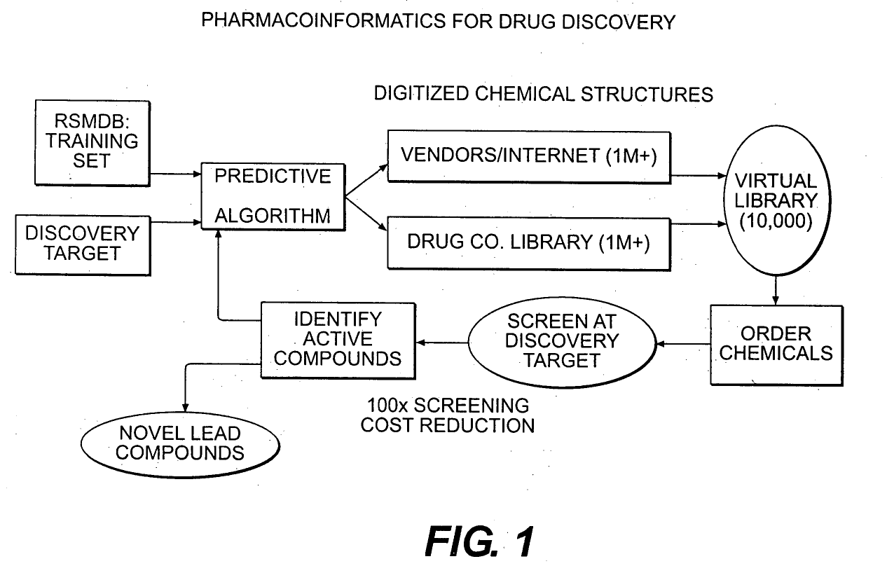

The process depicted in FIG. 1 entails in silico screening a million compounds and picking 10,000 for the confirmatory screen, for example. This in silico screen can be done in a matter of hours or days and reduces the cost of high throughput screening 99% because 100-fold fewer compounds are screened. The in silico screening process is only a predictive tool and is not 100% accurate. Nevertheless, enrichment of hit rates of several-fold to more than 80-fold has been demonstrated with this approach vs. random high throughput screening. Accordingly, the 99% cost reduction, while still achieving high hit rates, can give a tremendous boost to productivity and reduced costs for this phase of drug discovery. The huge cost savings should allow smaller drug discovery companies to compete effectively with larger drug companies in the discovery process.

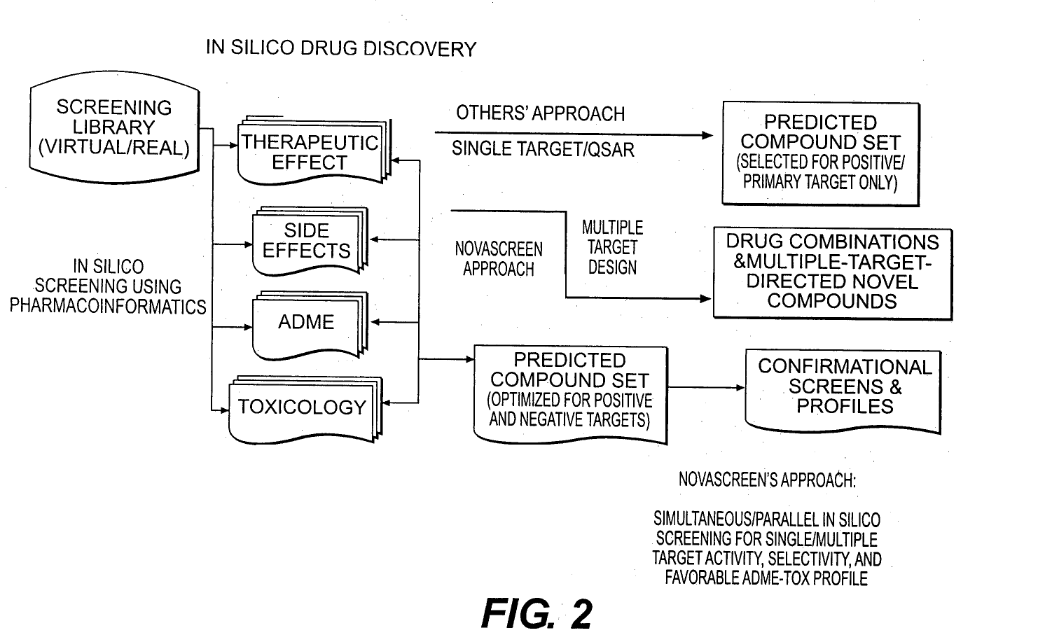

An even more powerful approach is to use pharmacoinformatics and in silico screening to predict molecular recognition by compounds at multiple targets simultaneously. For example, these tools can be used to search for chemical substructures that impart selectivity with respect to a specific subtype of a receptor class (for example, looking for compounds selective for the dopamine D1 subtype but not dopamine subtypes D2, D3, D4, or D5). Another example would be to identify or design drugs active at two or more targets at one time where the multiple targets are involved in the disease process. The ultimate objective is to identify or design drugs that act positively against one or more desired targets, do not recognize targets that cause side effects or toxicity, and have the chemical features for the desired oral absorption, metabolism and drug half-life, etc. In other words, designing new drug candidates from the earliest stages that would have a greatly enhanced probability of progressing all the way to FDA approval and market introduction without the current unacceptably high attrition rate. These advanced strategies are depicted in FIG. 2.

General Description of Data Interrogation Method and Gathering of Screening Compounds

The database, comprised of chemicals (and related information) and proteins (and related information) and measurements of interactions or lack of interactions, provided a set of data useful in drug lead discovery. As discussed previously, whether a chemical becomes a useful medication is ultimately determined by its overall molecular properties, that is the sum of activity profiles (PT).

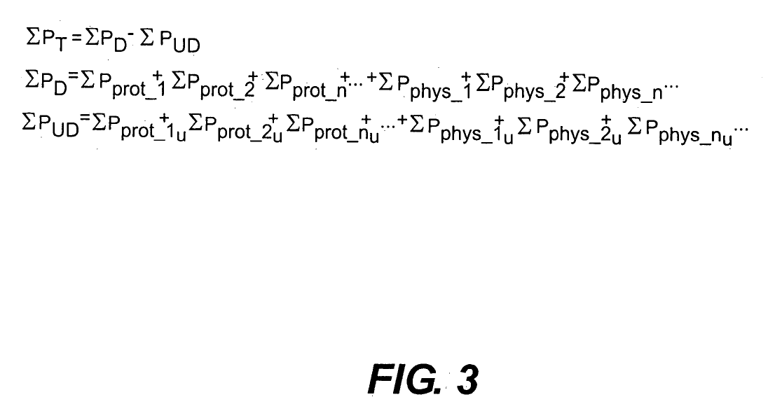

As shown in FIG. 3, the profiles of molecular properties may include activities with more than one protein (Prot_{—1 to n,} receptors or enzymes for instance) and exclude activities with proteins contributing to the unintended effects (Prot_{1u to nu}). The equation depicted in FIG. 3 defines the overall pharmacological profile (or molecular properties, PT) as “the sum of the desired properties (PD, activity profiles and physicochemical properties for instance) minus the sum of the activity that is undesired (P_{UD})”. Each term, either Pₚᵣₒₜ_{_{—}}₁ or Pₚᵣₒₜ_{_{—}}₁ᵤ, are a set of structural activity relationships derive statistically, that is each term P is a statistical presentation of a relationship between certain chemical descriptors and biological activity. The activity may be defined by different selection criteria. For example, a threshold of activity selection may be dependent on the natural testing sensitivity of assay and detection thresholds allowed by instruments used to characterize molecular interactions. The P_{phys}_{_{—}}ₙ are physicochemical properties of the molecule, and in certain instances, the P_{psys}_{_{—}}ₙ can be described in the same general term P where the physicochemical properties or parameters are part of the “descriptors” used for the structural activity relationship. Additionally, the sum of the profile may also extend beyond the realm biological activity to physical measurements and characterization ultimately affecting its biological properties.

For instance, in one of the later described examples (Example I), the so called desired molecular properties are defined as “activity with dopamine D1 receptors” whereas the undesired properties in part is defined as concurrent activity with an array of related membrane receptor and transporter. In Example II, the desired properties include the concurrent biological activity with two monoamine transporters; and in Example III, the desire molecular properties include activity with a pair of membrane receptors and lack of undesired activity with an assortment of targets as well.

These molecular properties are in fact structural activity relationships, which may be interrogated using different statistical tools. The following example uses identification of gathering a dopamine D1 biased chemical library as an example:

Goal of the Example: This example demonstrates an example of the method (and tools) that are useful (1) for the extraction of particular properties, structural-activity relationships and validate such relationship; and (2) demonstrating how to use such relationships to gather chemical libraries that are potentially biased for a particular activity or application. In this example, the desired properties are that the selected compounds must show preferred dopamine D1 inhibition characteristics and also lack of activity against 7 other receptors, D2, 5HT2A (serotonin), NET(norepinephrine transporter), AA2A (α-adrenergic 2a), AA2B (α-adrenergic 2b), AB1 (β-adrenergic 1) and AB2 (β-adrenergic 2). This parallel approach is quite challenging since all 8 eight targets are structurally correlated (see Table 1 below).

TABLE 1
Protein sequence Blast analysis between D1 and 7 other targets

| | Identity % | Similarity % |
|---|---|---|
| D2 | 48 | 63 |
| 5HT2A | 51 | 65 |
| NET | 30 | 55 |
| AA2A | 38 | 55 |
| AA2B | 42 | 63 |
| AB1 | 49 | 69 |
| AB2 | 32 | 62 |

The ideal compounds sought were potent binders with D1 with very weakly or no binding with the rest of 7 targets. To be able to achieve this goal, a multi-SAR relationship needs to be established to correlate molecular descriptors not only with D1 active data but also with 7 other receptors inactive data. The RSMDB has served perfectly for this purpose.

Data handling General Descriptions: The main data handing tool used in this project is ChemTree, which is a Recursive Partitioning (RP)-based algorithm and applied to analysis of 2-D bond length descriptor correlation with binding activity and also to screen virtue compound libraries. Strictly speaking, the molecular descriptor applied here is an approximate description since atom type, bending angle, and dihedral angle are not represented.

The QARSIS package was used as a tool to cross validate the predicted D1 active from ChemTree. The D1 radioligand binding measurements were perform against the compounds selected from in silico screening. About 6.5% of the compounds that exhibited activity of >50% inhibition at 10⁻⁵M concentration against D1. The 7 other assays were followed-up to check selectivity. Twenty-six compounds were identified as D1 selective inhibitors using the criteria that the compound's percent inhibition against seven other receptors are of 3 fold less compared to their D1 inhibition rate. Functional assays were applied to identify a compound's function in term of D1 cAMP signal.

Considering a traditional 0.1% hit rate for blind screening against one target, a 2.7% hit rate to obtain D1 selective inhibitors is very significant given the fact that there are high percentage similarities among those interesting targets. This may be the first successful attempt to consider multiple proteins (as many as 8) as simultaneous targets to screen compounds. The number of promising candidates may be expanded together with a few more key protein assays as filters against undesirable effects.

Training data set (a subset from the database used for the example) The so called training process is a process of using a existing dataset to extract or to identify chemical descriptors associated or unassociated with a biological activity. The training data set contains 1547 compounds and their inhibition results when screened against 8 receptors. The choice of percent of inhibition rather than Ki or IC50 is based on the fact that we need continuous data spectrum from inactive to active data. An example of the binding data are listed below as Table 2.

TABLE 2
Example of binding data used in the training set. The last
row is the number of compound with 50% inhibition
against each protein assay.

| ID | D1 | D2 | AA2A | AA2B | AB1 | AB2 | 5HT2A | NT |
|---|---|---|---|---|---|---|---|---|
| 1 | 0.06 | −0.07 | −0.33 | −0.03 | 0.06 | 0.02 | 0.08 | 0.52 |
| 2 | 0.94 | 0.24 | 0.82 | 0.75 | 0.00 | 0.11 | 0.88 | 0.25 |
| 3 | 0.49 | 0.97 | 1.00 | 1.00 | 0.75 | 0.58 | 1.04 | 0.08 |
| 4 | −0.01 | −0.06 | 0.57 | 0.32 | −0.08 | −0.05 | 0.05 | −0.09 |
| 5 | 0.04 | −0.04 | 0.28 | 0.00 | 0.14 | −0.03 | −0.01 | 0.08 |
| 6 | 1.03 | 1.01 | 1.00 | 1.03 | 0.50 | 0.75 | 1.08 | 0.96 |
| 7 | 0.87 | 1.00 | 0.99 | 1.03 | −0.02 | −0.04 | 0.72 | 0.85 |
| 8 | −0.04 | −0.05 | −0.21 | −0.11 | 0.07 | −0.01 | −0.12 | −0.04 |
| 9 | 0.01 | −0.11 | 0.27 | 0.24 | −0.20 | −0.16 | −0.05 | 0.06 |
| 10 | −0.03 | 0.06 | 0.06 | 0.16 | −0.02 | −0.03 | 0.09 | 0.03 |
| 11 | 0.31 | 0.43 | 0.71 | 0.78 | 0.17 | −0.15 | 0.17 | 0.13 |
| 12 | 0.03 | −0.17 | 0.06 | 0.06 | −0.14 | −0.04 | 0.02 | −0.03 |
| 13 | 0.95 | 1.03 | 0.82 | 0.99 | −0.19 | 0.03 | 1.08 | 0.02 |
| 14 | −0.06 | 0.00 | 0.11 | −0.10 | 0.04 | 0.09 | 0.04 | −0.01 |
| 15 | −0.06 | 0.03 | 0.14 | −0.17 | −0.23 | 0.09 | −0.04 | −0.11 |
| 16 | 0.10 | −0.02 | 0.08 | −0.01 | −0.12 | 0.01 | 0.07 | −0.06 |
| 17 | 0.24 | 0.16 | 0.24 | 0.81 | −0.04 | 0.02 | 0.59 | 0.69 |
| 18 | 1.04 | 0.75 | 0.91 | 0.94 | 0.26 | 0.02 | 1.04 | 1.00 |
| 19 | 0.44 | 0.93 | 0.79 | 0.85 | 0.01 | −0.08 | 0.10 | −0.02 |
| . . . | . . . | . . . | . . . | . . . | . . . | . . . | . . . | . . . |
| 1564 | −0.18 | 0.05 | −0.10 | −0.13 | 0.17 | −0.08 | 0.10 | 0.21 |
| 1565 | −0.09 | 0.07 | −0.19 | −0.01 | 0.15 | −0.16 | −0.09 | −0.08 |
| 1566 | −0.17 | 0.08 | −0.07 | 0.02 | 0.05 | −0.12 | 0.28 | 0.13 |
| 1567 | −0.22 | 0.22 | −0.13 | −0.11 | 0.00 | −0.08 | 0.30 | 0.19 |
| 1568 | −0.21 | 0.08 | 0.13 | 0.09 | 0.04 | −0.02 | 0.07 | −0.02 |
| 1547 | −0.17 | 0.21 | 0.09 | 0.17 | 0.05 | −0.09 | 0.12 | 0.63 |
| over 50% | 182 | 219 | 323 | 334 | 126 | 146 | 198 | 134 |

Cluster analysis was done on the traiming set compounds and biological data. Total 25 different chemistry classes were included within training set molecules. The largest cluster has 15 compounds and smallest one 2 compounds, which confirms the reasonable diversity of our training data set. Another input is the molecular structure file formatted as SDF files. Notice that the sequence of SDF shall be presented in the same order as in binding data file.

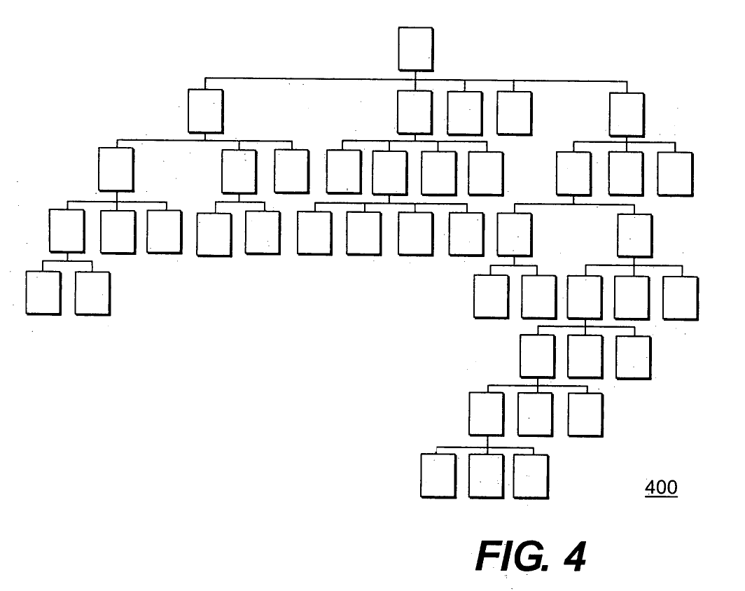

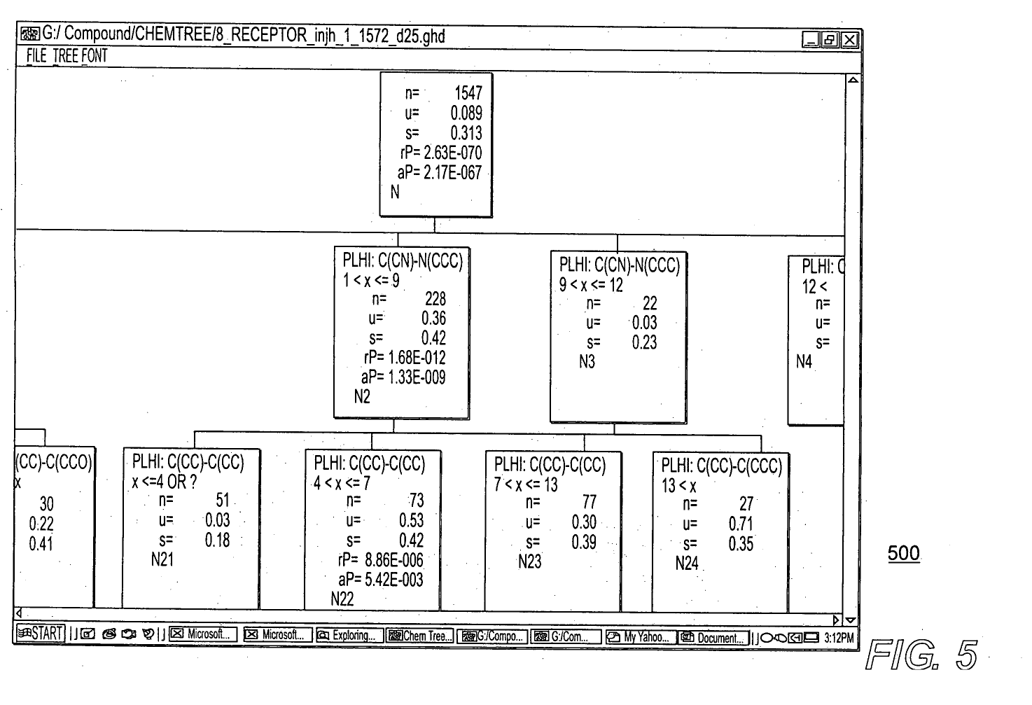

Method 1 (and example of data interrogation tool 1)—Application of ChemTree and RP algorithm—ChemTree package from GodenHelix Co is applied to classify compounds into a tree hierarchy for each protein according to RP algorithm. The complete D1 interactive tree image 400 is shown in FIG. 4.

Each black square in tree image 400 indicates a group of compounds classified by RP algorithms. The top one is termed the root since it contains all 1547 compounds and the lower squares are called nodes or leaves. Nodes which can not be further regrouped are called leaves, which means no statistical significance is found to further split this nodes compounds using defined 2D bond distance descriptors. Each node or leave represents group compounds with the same set of descriptors and also with average binding activity which is percent of inhibition in this study. An active leaf is defined as the average of percent of inhibition is above 50% and inactive leave as below 50%. Notice that compound screenings were performed using active leaves to maximize binding activities against the D1 receptor and using inactive leaves to minimize binding activity against the seven other receptors.

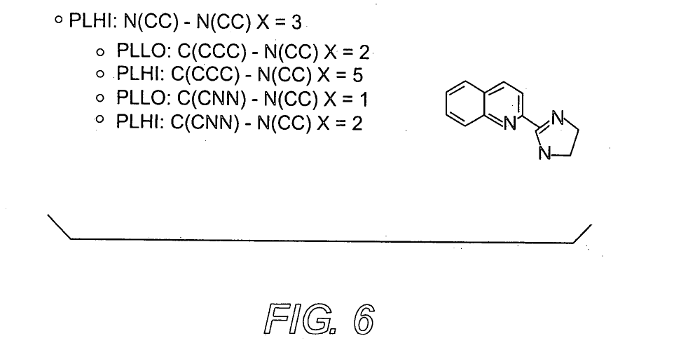

FIG. 5 depicts a partial D1 tree image 500. In the root of tree image 500, the square indicates 1547 compounds (n) in this entire tree and average percent inhibition (u) is 0.089 and standard deviation (s) is 0.31 and statistical indicator P-test values is 2.63E-70 for splitting downward. For each node or leaf, a descriptor and its value are arranged as shown, for example, PLHI: C(CN)-N(CCC) and 1<X<=9. This reads as, within that node or leaf, all compounds are of descriptor defined as: The bond number arranged between a Carbon atom which connects to C and N and a Nitrogen atom which connects to three C is from 2 to 9. Note that within each leaf, compounds not only share the same descriptor within leaves but also share the same group of descriptors defined at the nodes all way up to the initial root. An example of a 2D bond distance descriptors set is shown in FIG. 6.

Using ChemTree, chemical libraries (SDF and Mol files for instance provided by the chemical suppliers) may be searched using any of the nodes to find a list of compounds containing corresponding chemical descriptors. Using the “positive” nodes to search the chemical database, for example, one can compile a list of compounds containing the “positive” descriptors; hence these compounds are with higher probability of being active against the given biological targets. In contrast, using the “negative” nodes to search the chemical database, will lead to a list of compounds containing these “negative descriptors” and hence with a lower probability of being active against the given protein for the “known active chemical descriptors” of the training set that are “excluded”.

The probability differential, i.e. a higher probable activity with one target and a lower probable activity with other target of the same small organic molecule is the essence of the inherent small molecule selectivity. The core innovation and novelty is the use of the arrays of both positive and negative data in combination. Such a combination will be the principle guidance for the design of chemical libraries and selection of compounds to screens and ultimately the establishment of selective biological profile of small molecules.

For each of the compound libraries (compiled as one SDF file), the D1 tree will be applied first using the active leaves to screen compounds. Then the output SDF file of selected compounds will be screened against the D2 tree by selecting inactive leaves. Furthermore, the new output from D2 trees will be then put against the next receptor, 5HT2A. The same procedure will be repeated in sequence until all of the 8 receptors are screened.

Method 2 using QSARIS and its models—The QSARIS package is applied to confirmed D1 activities. A subset of the identical training data set is also used in ChemTree. Essentially, the input (449 compounds) data was employed to regress the correlation equation between D1 percent of inhibition and QSA RIS predefined descriptors, such as atom type E state, connectivity valence, H-bond, etc. Notice that only 2D descriptors were applied since our training data input SDF file is a 2D molecular description. The correlation was obtained as follows:

D1_INH=−0.08562*numHBa−0.8676*xch5+2.667*xch7+0.02839*SdssC+0.1488*SaaaC−0.2296*SssssNp+0.01234*SsF−0.1567*SsI+0.02749*SaasC_acnt−0.109*SaaaC_acnt−0.1194*SssssC_acnt +0.169*SdNH_acnt+0.08198*SsssN_acnt−0.02624*k1+0.04148*SHsNH2−0.01579*Gmax+0.1932*Hmin+0.007205*SHBint+0.003658*fw−0.002946*ncirc−0.0775005.

The notation and statistical indicators of the above equation are listed below:

numHBa: Number of hydrogen bond acceptors.

xch5: Simple 5th order chain chi index

xch7: Simple 7th order chain chi index

SdssC: E-State indices of ═C<

SaaaC: E-State indices of Carbon with three aromatic connections

SssssNp: E-State indices of >N+<

SsF: E-State indices of −F

SsI: E-State indices of −I

SaasC_acnt: Count of all Carbon with two aromatic and one single bond connections

SaaaC_acnt: Count of all Carbon with three aromatic connections

SssssC_acnt: Count of all >C<

SdNH_acnt: Count of all=NH

SsssN_acnt: Count of all >N−

k1: Kappa 1 (kappa shape indices)

SHsNH2: Sum of −NH2

Gmax: Largest atom E-State value in molecule

Hmin: Smallest atom hydrogen E-State value in molecule

SHBint: Sum of internal of Hydrogen bonds

fw: Formula weight of a molecule.

ncirc: the total number of all cycles in the molecular graph

Multiple R-Squared=0.6157

Standard error of estimation=0.2307

F-statistic=34.28

P-value=0

Multiple Q-Squared=0.5651

Cross validation RSS=25.78

QSARIS concluded that: The training set is well described by the regression equation, which is statistically very significant. Cross-validation shows that the constructed model can be used, with some care, to predict the value of D1_INH.

Note that Lipinsky drug-like compound rules may be optionally enforced using QSARIS, and all of the Phase I results have been filtered by Lipinsky's rules.

Properties (SAR/OSAR) Validations—Validations were done against D1 models obtained from both ChemTree and QSARIS. Additionally, 18 compounds which were not included in the training data set were selected with half of them as D1 active and half of them as not D1 active.

The results of validation are listed in below Table 3.

TABLE 3

| | | Predic_{—} | Predic_{—} |
|---|---|---|---|
| ID | Inhibition_meas | by_ChemTree | by_QSARIS |
| Cpd1 | 0.86 | 0.30 | 0.34 |
| Cpd2 | 0.99 | 0.83 | 0.72 |
| Cpd3 | 0.99 | 0.83 | 0.86 |
| Cpd4 | 0.98 | 0.30 | 0.68 |
| Cpd5 | 0.95 | 0.87 | 0.44 |
| Cpd6 | 0.99 | 0.83 | 1.01 |
| Cpd7 | 0.79 | 0.06 | −0.39 |
| Cpd8 | 0.94 | 0.87 | 0.45 |
| Cpd9 | 1.03 | 0.83 | 0.78 |
| Cpd10 | 0.07 | 0.06 | 0.85 |
| Cpd11 | 0.05 | 0.30 | 0.50 |
| Cpd12 | 0.33 | 0.13 | 0.36 |
| Cpd13 | 0.34 | 0.05 | 0.45 |
| Cpd14 | 0.22 | 0.05 | 0.35 |
| Cpd15 | 0.17 | 0.06 | −0.23 |
| Cpd16 | 0.28 | 0.06 | −0.39 |
| Cpd17 | 0.29 | 0.83 | 0.24 |
| Cpd18 | −0.09 | 0.13 | 0.19 |

Eighteen (18) compounds were queried against the D1 target. The second column is obtained from real measurement and the third and fourth columns are representative for predicted inhibition against D1 using ChemTree and QSARIS, respectively. For the active compounds, 6 out of 9 compounds were predicted by ChemTree and 5 out of 9 were predicted by QSARIS with inhibition above 50%. For the inactive compounds, 8 out of 9 were predicted by both ChemTree and QSARIS with inhibition below 50%. This demonstrates that the prediction process using in silico screening is in reasonable agreement with experimental results and confirmed that our SAR models can provide reasonable screening results.

General Description of “Screening” Compound Selection (Compound libraries and its sequential screening in the 8 biological target list)—Eleven compound libraries were processed using above described in silico screening methods, namely, ASINEX, ChemDiv, Enamine, ComGenex, Would Molecule(MDD), MayBridge, RCL, Imation, IBX, SPECS, WSB. First of all, D1 and then the remaining 7 other targets were screened (done sequentially). ChemTree models were applied to cherry picking compounds. Secondly, QSARIS's models and Lipinsky's rules were used to further screen compounds. The obtained compounds were also further filtered by kicking-out too closely similar compounds using compound diversity analysis. From the vendor's confirmation, over 1000 compounds were selected to purchase with the finally delivered compounds numbering 961. These 961 compounds were diluted and placed in either 96 wells or 48 wells plates for radio-ligand binding screening.

Applications of Pharmacoinformatics Technology for Drug Discovery Methods

Drug discovery and development strategies and methods can be designed to optimize the chance of success, reduce the risk of failure, and minimize the development time and cost using the pharmacoinformatics technology for the following broad applications:

1. Identifying new therapeutic applications of marketed pharmaceuticals or other 1 compounds with demonstrated safety primarily directed toward proven drug targets or combinations of drug targets for a specific therapeutic application; derived directly from the RSMDB dataset.

2. Discovering combinations of marketed drugs for complex diseases and multiple sites or targets; derived directly from the RSMDB dataset.

3. Selecting new chemical entities to address unmet needs against single or multiple proven drug targets; based on in silico screening of accessible compound libraries.

4. Developing new chemical entities against novel but validated drug targets;

derived from in silico screening of accessible compound libraries and medicinal chemistry.

5. Designing new chemical entities against proven or novel targets; based on in silico screening, medicinal chemistry, and/or de novo drug design.

Target Selection Criteria

Molecular targets for drug discovery can be generally classified into four categories:

1. Validated targets against which effective therapeutic agents are currently approved for medical use;

2. Targets related to market-validated targets (such as receptor subtypes) for which currently approved drugs may or may not be approved;

3. New biologically-validated disease targets for which approved drugs are not yet currently available; and

4. New targets (including “orphan receptors”) identified from genomics programs but for which the disease relevance is not yet known and no drugs are available.

Drug development risk increases successively with each of these four groups. There is a substantial opportunity for identification and development of new and improved drugs for the first two categories with substantially reduced development costs and risk profile vs. the latter two groups. The pharmacoinformatics technology, however, is applicable to all four categories.

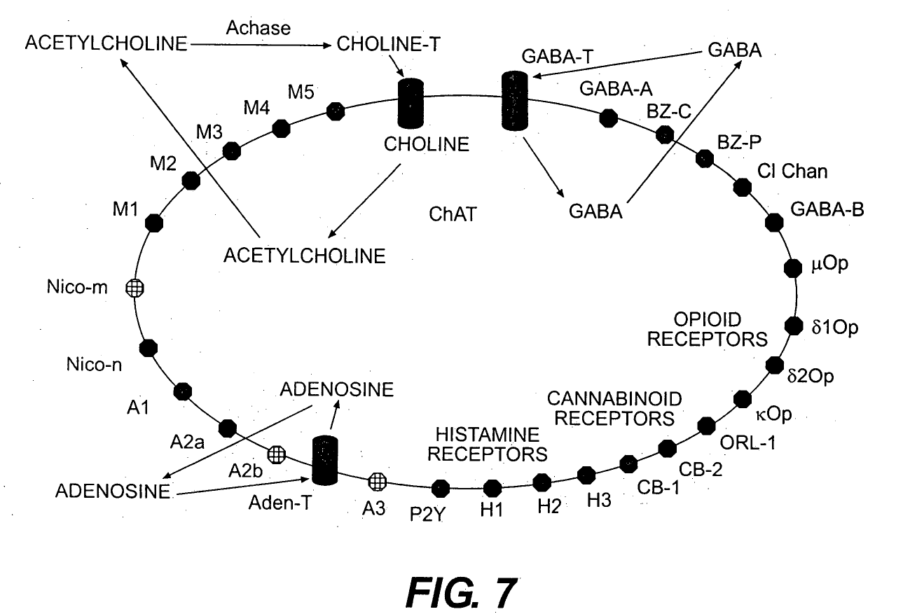

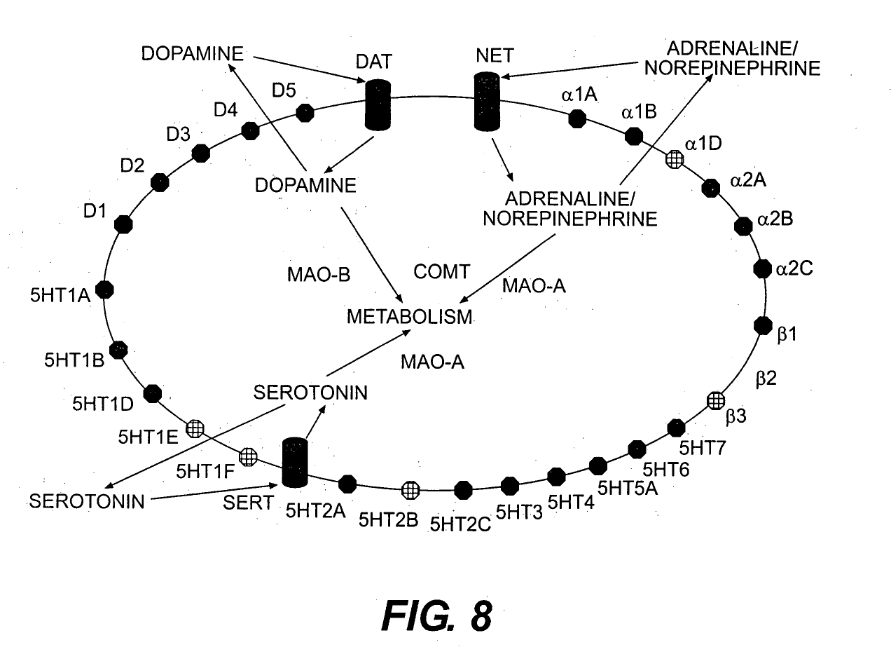

More than 50% of all drug sales, representing a worldwide market segment of at least $175 billion, are based on agents that act at G-protein coupled receptors (“GPCR's”). With about 70 such molecular targets in one embodiment of the RSavDB, the pharmacoiriforw-iatiacs platform can cover nearly all major types of GPCR classes, as well as most of the subtypes of these receptor classes. Selected classes of these receptors, and biologically related targets such as transporters or receptor-linked channels, can be a primary focus of drug discovery programs. These classes and related targets include the following receptor/transporter systems: dopamine, serotonin, and adrenaline/norepinephrine (adrenergic) (see FIG. 8), as well as GABA, opioid, adenosine, acetylcholine/muscarinic/nicotinic, cannabinoid, and histamine (see FIG. 7). Many of these receptor classes represent sites of action for drugs of abuse, and the same receptor classes are relevant to other critical medical needs that address very substantial markets with unmet needs, especially for treating central nervous system diseases or conditions, including psychiatric diseases, drug addictions, neurodegenerative diseases, and similar areas.

Representation of GABA (GABA-T, GABA-A, BZ-C, BZ-P, CI Chan, GABA-B), acetylcholine (muscarinic and nicotinic) (choline-T, M1-M5), adenosine (A1, A2a, A2b, Aden-T, A3, P2Y), histamine (H1-H3), cannabinoid (CB-1, CB-2), opioid (μOp, δ1Op, δ2Op, κOp, ORL-1), receptor classes and subtypes (octagons), and transporters or reuptake sites (cylinders) are shown in FIG. 7. All available subtypes are displayed. Solid colors are NovaScreen assays/targets in RSMDB; checked are under development or not yet available. Also shown is the key enzyme acetylcholinesterase (Achase) that converts the neurotransmitter acetylcholine to choline for reuptake, which is a NovaScreen assay too.

Representation of dopamine (DAT, D1-D5), serotonin (5HT1A, 5HT1B, 5HT1D-5HT1F, SERT, 5HT2A-5HT2C, 5HT3, 5HT4, 5HT5A, 5HT6, 5HT7), adrenaline/norepinephrine (adrenergic) (NET, α1A, α1B, α1D, α2A, α2B, α2C, β1, β2, β3), receptor classes and subtypes (octagons), and transporters or uptake sites (cylinders) are shown in FIG. 8. All available subtypes are displayed. Solid colors are NovaScreen assays/targets in RSMDB; checked are under development or not yet available. Also shown are key enzymes (monoamine oxidase A—MAO-A; monoamine oxidase B—MAO-B; and catechol-o-methyl transferase—COMT) that metabolize the neurotransmitters dopamine, serotonin, and norepinephrine, each of which are NovaScreen assays too.

Enzymes are another important category of proven drug targets, accounting for an estimated 21% or $66 billion of pharmaceutical sales worldwide. Enzyme inhibitors are especially important for antibiotics, antiviral agents, and anticancer drugs. Targets in these areas can be an additional focus of drug discovery efforts using pnarmacoinformatics databases and methods.

Compound Selection Criteria

Compounds for drug discovery and development can be generally classified into four categories:

1. Marketed pharmaceuticals approved for specific indications in the U.S. or elsewhere that have a proven, acceptable safety and pharmacokinetic profile and may (proprietary drug) or may not (generic drug) have currently valid patents on their structure;

2. Discontinued drug candidates or other known compounds such as agrichemicals or veterinary drugs that may have a proven, acceptable safety and pharmacokinetic profile based on prior animal and/or human testing and may or may not have currently valid patents on their structure;

3. Known compounds from industry sources but with unknown specific activities that can directly become lead compounds or new drug candidates or form the basis of pharmacophores or base chemical structures to derive novel chemical compounds; and

4. Novel chemical structures that are previously unknown and can be designed de novo and synthesized as potential new chemical entities (NCE) for drug development.

Again, drug development risk increases successively with each of these four groups, although the value of the compounds may increase through each category as the strength of the intellectual property position grows. Small molecule drugs that ultimately prove useful as orally-active pharmaceuticals must meet certain criteria with regard to efficacy, safety or side effects, and pharmacokinetics. Unfortunately, starting with novel, previously untested compounds, the failure rate is extremely high (>90% of compounds entering preclinical/clinical development never reach the market). The power of the pharmacoinformatics platform allows one to select and design new chemical entities (groups #3 and #4) with enhanced probability of success using information on chemical substructures or descriptors and molecular recognition algorithms using the databases.

A substantial opportunity exists for identification and development of new therapeutic uses of approved drugs and rescue of discontinued drug candidates or of compounds such as agrichemicals used for other purposes (categories #1 and #2 above) for new indications by harvesting the direct drug-target reactivity data in the RSMDB. In the event such drugs or discontinued drug candidates are patented entities, “use” patents for the new applications may be obtained. In the case of generic drugs, use patents should lead to an exclusive market position in the new field for the drug. With a majority of drugs off patent, and many older drugs never having been so broadly tested for reactivity with molecular targets as has been done with the RSMDB, there is a unique opportunity to uncover new uses of old drugs with far lower development risk, lower costs, and shorter time to market. Numerous precedents for this approach exist, including sildenafil (Viagra; Pfizer), which was originally developed for cardiovascular disease and later became a blockbuster drug for treating male impotence. Other examples are minoxidil (Rogaine; Pharmacia Upjohn), which was developed to treat hypertension and gained greater success as a hair growth stimulant for baldness, and amantadine (Symmetrel, DuPont Pharma), which was developed as an antiviral agent but later found to be effective in treating parkinsonism (tremors).

### EXAMPLES

Drug discovery and development programs, for example, can be focused on a broad category of molecular targets that are central to both treatments (1) for drug addiction and (2) for a wide range of central nervous system disorders. The drug addiction program can be centered on two groups of molecular targets (dopamine, serotonin, and norepinephrine transporters, and dopamine receptors) for treatment of cocaine addiction and one molecular target class (GABA-A/benzodiazepine receptors) for treatment of barbiturate (sleeping pill) addiction. These same two groups of molecular targets for treating cocaine addiction (neurotransmitter transporters and dopamine receptors) are also relevant to treatment of depression, attention deficit hyperactivity disorder, and obesity (transporters); schizophrenia, epilepsy, and Parkinson's Disease (dopamine receptors), and other CNS diseases. The target area (GABA-A) for barbiturates is also important for drugs to treat anxiety (sedatives), prevent convulsions, induce sleep, and as muscle relaxants. Treatments for Parkinson's disease can also be focused on adenosine receptor subtypes together with dopamine receptor subtypes. Numerous other drug discovery and development programs that address receptors, transporters, ion channels, and other ligand-Dinding molecular targets for a wide range of diseases can be designed using this technology platform.

An additional drug discovery and development effort can be focused on selected enzyme-based molecular targets that are associated with certain biochemical mechanisms of bacterial infections, viral infections, and cancer. Each of these programs can involve novel targets that have known involvement in disease-related processes. One program could involve a family or different families of enzymes for which certain forms (isozymes) mediate spread of tumor cells (metastasis) or are involved in other disease processes, and other forms provide necessary normal functions in the body. Pharmacoinformatics and in silico screening can be used to identify compounds that selectively block the metastasis-related or other disease-related isoform(s) and not the beneficial forms.

### Example 1

### Compounds Active at Both of Two Therapeutic Targets and Inactive at One Related Target for Cocaine Addiction Medication—Direct Database Interrogation

Scientists have learned much about the biochemical processes involved in the human brain related to such basic behaviors as pleasure, reward, excitement, fear, anxiety, sleep, etc. Central to these phenomena are the release from nerve cells, the extracellular activity, and the reuptake back into nerve cells of a group of neurotransmitter chemicals called catecholamines, which include dopamine, serotonin, and norepinephrine. The extracellular activity of these chemicals is primarily mediated by binding of the neurotransmitters to cell surface receptors, and the reuptake is accomplished by transporters that bridge through the cell membrane. Receptors for the neurotransmitters exist in numerous forms, or subtypes, and are distributed in different tissues and organs in the body.

Substances that make humans feel good all have a remarkably similar effect on a region of the brain called the “pleasure” or “reward” center. Nearly all of these substances have the capacity to increase the levels of dopamine in the nerve synapses in the “pleasure” center of the brain. Some substances have a direct effect on dopamine, others have an apparent indirect effect mediated by interactions between the substances and other types of receptors and transporters. The end result is the same, however. The feeling of pleasure resulting from the heightened levels of dopamine can lead to the behavior of “reward” by continuing to feed the brain with the pleasure-inducing substance to maintain the high dopamine levels. This is the essence of addiction. The pleasure inducing substance can be cocaine, heroin, amphetamines (speed), or any number of other drugs of abuse or they can be pharmaceuticals intended to have other beneficial effects, or they can even be genetic, environmental, or behavioral factors themselves.

While the end result is basically the same, the means is different. Blocking drug addiction for specific substances therefore requires an understanding of the complex mechanisms and interactions leading up to the elevated dopamine levels. Furthermore, since the perturbations associated with addiction are associated with effects common to a wide range of emotional or behavioral factors associated with numerous CNS diseases, understanding this complex set of targets can form the basis of finding improved drugs for treating diseases that represent enormous markets. Since the RSMDB can contain nearly all of the known molecular targets in this array of dopamine/serotonin/norepinephrine targets, as well as numerous other drug addiction primary targets (such as the GABA, opioid, and cannabinoid receptors), and because a wide array of CNS drugs have been screened for their selectivity at these targets to create datasets for the RSMDB, the pharmacoinformatics platform technology is uniquely capable of addressing these important therapeutic areas.

The war on drugs is consistently ranked as an initiative that should be one of our nation's highest priorities. Cocaine, a drug extracted from the coca plant and one of the most addictive drugs of abuse known, is an especially important concern. More than 23 million Americans have used cocaine at some time in their lives, of which an estimated 1.4 million are regular cocaine users. A similar number of regular users is estimated for Europe. Cocaine has potentially life-threatening effects on the cardiovascular system and causes long-lasting, adverse behavioral modification. Illegal drug use is estimated to cost our nation $67 billion annually in terms of lost productivity and treatment. A medication to treat cocaine abuse and dependence is an unmet need and one of the nation's highest priorities for development. An urgent need now exists to develop therapeutic compounds that reduce drug craving, block withdrawal symptoms, and prevent relapse.

There is currently no drug on the market in the United States for treating cocaine addiction. One drug, methadone, is approved for treating heroin addiction and costs approximately $300-600 per course of therapy. The potential market for an effective drug for treating cocaine addiction is estimated at about $1 billion based on 3 million regular users in the U.S. and Europe and pricing comparable to that for methadone treatment.

Drugs of abuse such as cocaine are known to interact with specific neurotransmitter-related receptors or transporters on the surfaces of cells located in the brain. For example, cocaine has been shown to directly affect the transporter and receptors for the neurotransmitter dopamine, and specifically to block the dopamine transporter. As noted above, these interactions are believed to mediate the biological activity and/or the mechanism of addiction of drugs of abuse. In the case of cocaine, there is a direct effect on dopamine levels in the “pleasure” center of the brain, which probably accounts for the strong addictive nature of cocaine. Compounds that interfere with or prevent interactions between cocaine and certain receptors or transporters in the brain may therefore have significant therapeutic potential to combat abuse and addiction.

In one embodiment of the RSMDB, a dataset of the molecular target interactions of a library of known addictive substances was established in order to predict molecular recognition patterns that may be associated with addiction. One such addictive compound that was tested was cocaine, which was profiled for potential activity or reactivity against more than 130 different molecular targets. From this Cocaine and Drug Addiction Database, a number of other targets were identified at which cocaine demonstrated activity, in addition to the known effect of cocaine on dopamine transporters. These key discoveries form the basis of programs to develop drugs for treating cocaine addiction and other chemical dependencies.

Cocaine Addiction—Neurotransmitter Transporter Agents

Through the Cocaine and Drug Addiction Database, other neurotransmitter transporters in addition to the dopamine transporter (DAT) have been identified that appear to play a key role (positive or negative effect) in cocaine addiction. These include the serotonin transporter (SERT) and norepinephrine transporter (NET). A discovery program for cocaine addiction medications can be based on compounds that block, partially block or fail to block, in a certain balance, DAT, SERT, and NET. The compounds fall into three categories: (1) single agents identified from the RSMDB that fit the specified balance and are known compounds with proven safety profiles; (2) combinations of two such compounds (known with safe profiles) identified from the RSMDB that together bridge the specified ratios at SERT, DAT, and/or NET and can be used as a cocktail for treating addiction; and (3) new agents or combinations of new agents with optimized activities according to the specified desired balance at DAT, SERT, and/or NET discovered through the use of the RSMDB and in silico screening methods. Each of these approaches seek agents that demonstrate (1) absence of abuse liability, (2) suppression of the acute reinforcing effect, and (3) reduction of withdrawal symptoms and craving.

(1) Single agent therapy with known compounds. A series of compounds have been identified from the RSMDB that demonstrate the desired activities for DAT, SERT, and NET. These include NBC-39900, NBC-72210, and NBC-59310, as well as NBC-71000 and NBC-26210, which are both active ingredients in generic medications approved by the Food and Drug Administration (FDA). Accordingly, these compounds have a proven record of safe use in humans and are being tested in animal efficacy models of cocaine reward behavior.

(2) Drug combination therapies with existing medications. Drug combination therapies developed to treat cocaine addiction may exhibit advantages over single agent approaches. It circumvents the “magic bullet” approach, and calls for a more dynamic approach, an “adjustable” drug-combination therapy. Our Cocaine and Drug Addiction Database indicates that cocaine addiction is likely the consequence of cocaine's blockage activity at DAT and SERT rather than any one of the transporters alone. Treating cocaine addiction may need to be based on finding “functional antagonists” at both transporters, but where the effect may need to be separable. Combinations of such drugs will ideally have effects on both DAT and SERT and such effects could be titrated or “attenuated” to gradually “wean the patient off” the illicit drug effects or chemical dependency. Discovery of such differential and complementary activity by two sets of compounds would be an extraordinarily difficult and costly R&D effort under traditional in vitro screening paradigms. Using the RSMDB, however, we have identified a series of compound combinations that meet these criteria and are entering animal efficacy studies.

### Example II

### Method of Identifying Compounds Concomitantly Disrupting the Activities of a Pair of Monoamine Transporters (Inhibition of Dopamine and Serotonin Re-Uptakes, a Method Offinding Compounds Useful in Medication Development of Medication for Cocaine Addiction, ADHD, and Cognitive Disease Managements)

Rationale of Target Composition and Technical Background

Recent reports indicate that brain levels (concentration) of both dopamine and serotonin are related to the cocaine addiction. Description of the importance of concurrent inhibition of dopamine and serotonin re-uptake activity is described in U.S. application Ser. No. 10/105,407, fled Mar. 26, 2002, which is incorporated by reference. An independent study using double transporter knock-out animal model further confirmed the observation that was obtained from a comprehensive profile of cocaine.

Using double transporter knock-out mouse models, it was pointed out that (1) cocaine may normally work to provide rewarding action at both dopamine and serotonin transporters; and (2) Either dopamine or serotonin transporter can mediate cocaine reward in the life long absence of the other transporter (more information may be found in Sora et al, “Molecular mechanisms of cocaine reward: Combined dopamine and serotonin transporter knockouts eliminate cocaine place preference,” PNAS, Apr. 24, 2001, which is hereby incorporated by reference). This observation is critical for developing treatment of cocaine addiction and craving.

From the above observation, one may ascertain a scientific hypothesis that the enrichment of brain dopamine/serotonin levels by concurrent blocking of the reuptake sites of dopamine and serotonin with a combination of transporter selective chemicals, or affecting them with a single chemical entity may help to ameliorate certain symptoms of the addiction. Thus, a goal is to find clusters of organic small molecules specifically affecting either, the DAT or SERT monoamine transporters, or simultaneously affecting both DAT and SERT. These compounds will also be demonstrating pharmacological profiles which make them suitable to be used as research tools to assess the possibility of abolishing symptoms of cocaine addiction in an animal model. The lead compounds identified are useful in validating the hypothesis stated previously.

Experimental Approaches

Step 1. Design chemical libraries (based on existing SAR models) with a “statistical” propensity to be selectively reactive with the dopamine transporter, or the serotonin transporter or both transporters simultaneously.

Step 2. Optimize the virtual chemical collection of Task 1 by identifying chemical descriptors (negative descriptors) devoid of receptor activities at beta adrenergic receptor subtypes β1, β2, and β3, muscarinic receptor subtypes M₁, M₂, M₃, M₄ and M₅;

Step 3. Acquire chemical libraries of approximately 800 compounds (selected from >2.5 million compound library) with “clustered” potential activities against dopamine-serotonin;

Step 4. Profile the compound collections using in vitro radioligand binding assays.

Example of Tissue Based Transporter Functional Assays

SEROTONIN UPTAKE (Human) ASSAY, [³H]-5HT Uptake Using Human Platelets

Tissue Preparation

1. Harvest platelets by decanting cells and media into 50 ml conical tubes.

2. Centrifuge in a Sorvall table top centrifuge at 1500 RPM for 10 minutes at room temperature.

3. Decant about 80% of the supernatant into bleach, leaving the rest with the pellet.

Gently resuspend each pellet to its original volume with the addition of Krebs-Ringers-HEPES (KRH) buffer. This initial concentration (I.C.) equals approximately 2.5×10⁶ cells/ml, so that the final concentration is 0.5×10⁶ cells/tube, or 2×10⁶ cells/ml.

Reaction

1. Each tube or well receives the following components: 25 μl drug or vehicle; and 200 μl cell suspension.

2. Incubate the above mixture for 15 minutes at room temperature. Initiate the uptake reaction with the addition of: 25 μl [³H]-5HT (5-hydroxytryptamine), and incubate for 15 minutes at 37° C.

3. Terminate the reaction by dilution of the assay tube contents with ice-cold saline, followed by rapid vacuum filtration of the assay contents onto untreated GF/B filters.

4. Wash the tubes and filters 5 times with 1 ml of cold saline.

5. Radioactivity trapped onto filters is assessed using liquid scintillation spectrophotometry after soaking the filters for at least three hours in scintillation cocktail.

Materials and Reagents

1. [[³H]-5HT is diluted to 300 nM in KRH, such that the final substrate concentration in the assay is 30 nM. Table 4 shows the composition of theKRH buffer.

2. Non-specific uptake is defined as that remaining in the presence of 1×10⁻⁶ M imipramine.

3. The reference compound is imipramine run at final concentrations of; 1×10⁻¹⁰, 3×10⁻¹⁰, 1×10⁻⁹, 3×10⁻⁹, 1×10⁻⁸, 3×10⁻⁸, 1×10⁻⁷ 3×10⁻⁷, 1×10⁻⁶ M.

4. The positive control is imipramine run at final concentrations of 1×10⁻⁸, 3×10⁻⁸, and 1×10⁻⁷ M.

TABLE 4

| Krebs-Ringers-HEPES | M.W. | g/250 ml |
|---|---|---|
| 125 mM NaCl | 58.4 | 1.825 |
| 4.8 mM KCl | 74.6 | 0.09 |
| 1.2 mM KH₂PO₄ | 136 | 0.04 |
| 5.6 mM glucose | 180 | 0.25 |
| 0.5 mM EDTA | 372 | 0.047 |
| 25 mM HEPES | 238 | 1.5 |

Results

Using conventional methods, such a multiple-targeted discovery goal is difficult to achieve. For instance, conventional high throughout screening often gives a “hit-rate” of 0.1%. The probability of finding a compound with dual-functionalities, as DAT-SERT, is of the order of a few in a million.

In this example, in order to find compounds with designed profiles of activity, we institute a simple approach using sequential in silico screening utilizing the existing proprietary dataset (RSMDB) and existing SAR. The dataset, unlike those compiled from public literature, is an internally consistent full rank data matrix. For example, the outputs of the activity profile of a chemical or a biological, active and inactive, are accurate reflections of their overall in vitro chemicallbiological activities; whereas the data compiled from the public domain (1) does not indicate negative information, and (2) are lack of internal consistency for the different assaying methods and platform used even with one specific molecular target. Using the full rank dataset, one may wish to derive, for instance, biological profiles of particular chemical descriptors (2D or 3D structural components) found to be linked with or devoid from any biological activities. Regardless of the method of data interrogation, these chemical descriptors will represent a “true” reflection of their associated biological profiles.

The primary statistical clustering method used in this example is based on recursive-partitioning (RP). We use RP to interrogate the dataset and to derive structural activity relationships (and structural-inactivity-relationships). The advantage of this algorithm is its ability to handle the coexistence of a multitude of SARs, and the ability to sort and group these relationships accordingly. Moreover, it has the ability to model and forecast nonlinear SARs, which are common phenomena. We primarily rely on a commercial software package, ChemTree (GoldenHelix). In general, statistical clustering is often more superior and versatile than other data handling algorithms. Such versatility is more pronounced when dealing with “activity” data that could be contributed by diverse class of chemicals, multiple mode of activities (agonists, antagonists, partial agonists, inverse agonists etc), and different orientation of molecular interactions, which as often the case with chemical activity data set of GPCR receptors. This versatility can also be reflected in its ability to separate chemical descriptors associated with a particular activity from those descriptors that are devoid of same activities.

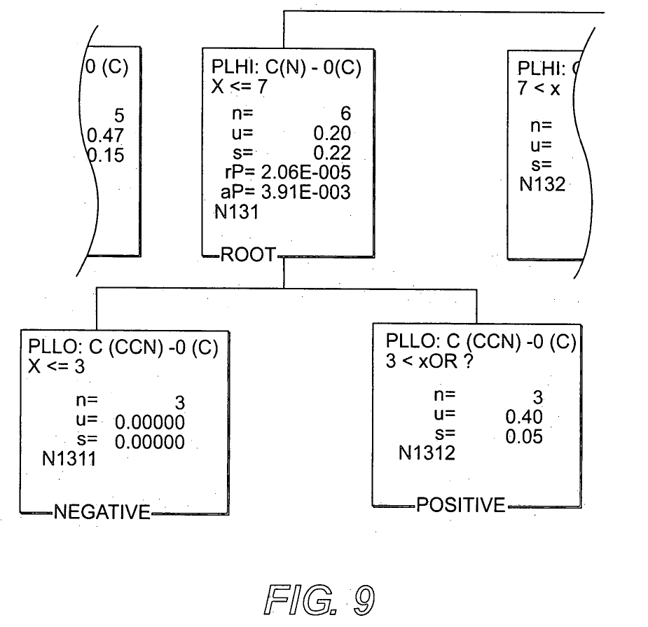

FIG. 9 represents a typical case of using recursive partitioning to identify chemical descriptor associated (positive)/unassociated (negative) with particular activities. Using the descriptors that are associated with certain biological activity, active compounds are likely to be found; whereas using descriptors devoid of such associations will likely lead to inactive compounds. In FIG. 9, The top node is a root containing six compounds. Using p-test, the root is further split into chemical with a given descriptor contributing to the observed activity (positive) and descriptors unassociated with the observed activity (negative).

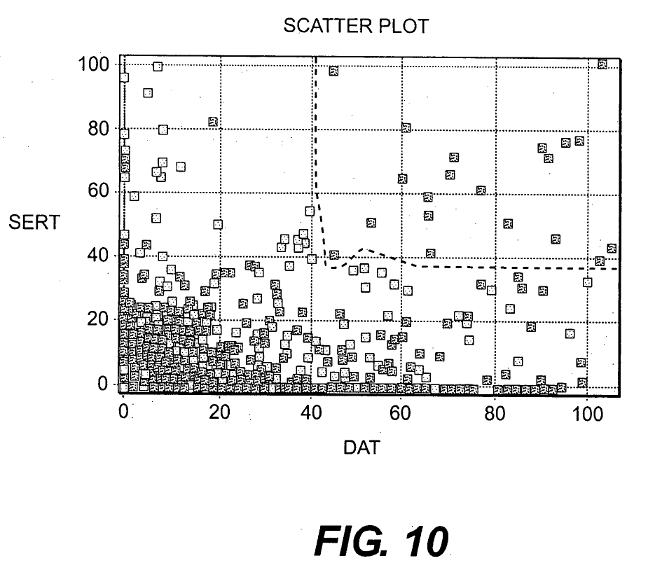

To find chemicals that are active at multiple biological targets, each of the multiple structural-activity clustering in sequence may be used. FIG. 10 demonstrates a result of trying to find a compound active against two monoamine transporters, DAT and SERT. FIG. 10 shows a partial dataset representing the optimized probability of finding compounds modulating activities at multiple biological targets (DAT, x axis; vs. SERT, y-axis). The “dots” in the upper right hand corner of the graph (to the right of and/or above the dotted line) are those found to be active with both transporters. A few demonstrated potency in nM with the respective transporters.

Two clustering “trees” were built from the existing dataset; each was from a data set of particular transporters, and each produced a set of active (positive) descriptors. One set of (DAT related) “positive” descriptors were first used to “scan” a chemical database; a population of compounds were found that were “carriers” of these descriptors. Another set (SERT related) of “positive” descriptors were then used ti “scan” those DAT positive descriptors “carriers”, from which a sub-population of compounds were found that were carriers of both DAT and SERT positive descriptors. A subset of this population were subsequently tested. From a rather scanty library (<1,000 compounds) quite a few compounds were identified demonstrating potent inhibitory activity against the reuptake of both monoamine transporters. This was a significant improvement over the “yield” on conventional random screening (expected yield of finding a single chemical entity active against 2 biological targets is 1/1,000,000).

### Example III

### Compounds Active at Both of Two Therapeutic Targets and Inactive at One or More Related Targets for Other Therapeutic Indications—In Silico Screening Methods for New Compound Discovery

Drugs for Depression, ADHD, and Obesity

The cocaine addiction treatment program based on the selectivity ratios of compounds for SERT, NET, and DAT may be used, along with the pharmacoinformatics technology platform, to identify new, safer and more efficacious compounds for treating depression and disorders such as attention deficit hyperactivity disorder (ADHD) and obesity. Several potential candidates, including some compounds with a demonstrated record of safety, have been identified from our RSMDB, and efforts to find new chemical entities through in silico screening are being pursued.

Depression is one of the most common psychiatric disorders, with estimates that at any one time 5%-6% of the population is depressed, and 10% suffer depression at some point in their lifetime. Antidepressants are a very large market, estimated at $14 billion worldwide. Therapeutic indications within the category of antidepressants include depression (including manic depression or bipolar disorder), panic disorder, obsessive-compulsive behavior, eating disorders (obesity and anorexia), and attention deficit hyperactivity disorder. Some antidepressants (tricyclics) are also used to treat enuresis/incontinence and chronic pain.

One of the predominant modes of action of antidepressants is the inhibition of transporters or reuptake sites for dopamine (“DAT”), serotonin (“SERT”), and norepinephrine (“NET”). The earliest antidepressant drugs, called tricyclics, work primarily by inhibiting both SERT and NET. A later generation of more specifically targeted drugs are the selective serotonin reuptake inhibitors (SSRIs), exemplified by fluoxetine (Prozac; Pfizer), which blocks SERT preferentially and captured a dominant market share af ter its introduction. More recently, venlafaxine (Effexor; American Home Products) has been gaining market share based on its profile of activity, which includes greater selectivity towards NET. Both of these classes of drugs exhibit interactions with other molecular targets that may mediate some of the numerous side effects of antidepressants. Furthermore, two-thirds of patients suffering from depression fail to respond to existing drugs. Clearly, large market opportunities still exist in the antidepressant market for new agents that exhibit improved efficacy or safety based on their relative potency at key targets such as SERT, NET, and DAT and their overall selectivity. Other classes of antidepressants are (i) compounds that are inhibitors of the enzyme monoamine oxidase (MAO), a target that is also in the RSMDB, and (ii) certain heterocyclic compounds, such as bupropion (Welbutrin; GlaxoSmithKline), which have unique modes of action that may include blocking serotonin receptor subtypes such as 5HT2a, 5HT2c, or 5HTla, and/or adrenergic receptor subtypes such as alpha2. All of these receptors are included in the RSMDB, as well. Using the RSMDB and in silico screening strategies, new and more effective antidepressants or related drugs can be designed that address two or more relevant targets in a positive manner, and new and safer therapeutic agents in these areas can be designed by identifying compounds with desired activity at one or more targets and little or no activity at targets associated with side effects or other adverse properties.

### Example IV

### Compounds Active at One Therapeutic Target and Inactive at a Multiplicity of Potential Side Effect Targets for Cocaine Addiction Medications or Treating Parkinson 's Disease—In Silico Screening Methods for New Compound Discovery

Cocaine Addiction—Dopamine Receptor Subtype Selective Agents

Although the Cocaine and Drug Addiction Database identified DAT and SERT as the key targets for cocaine activity, there is evidence that secondary effects of cocaine and potentially other addictive substances are mediated through receptors for dopamine. Dopamine receptors exist in five different variations, or subtypes, called D1, D2, D3, D4, and D5. Each of these dopamine receptor subtypes may have different distribution patterns in the body and different reactivity or molecular recognition patterns correlated with the binding of ligands or other chemicals. Therefore, finding subtype-selective chemical compounds is an important goal for drug discovery, and the pharmacoinformatics technology is ideally suited for this type of activity.

In the case of cocaine addiction medication development, the initial emphasis is on selective agents for the dopamine D1 receptor. The power of this approach is demonstrated by the results of the initial in silico screening program. We used the RSMDB to generate predictive algorithms describing chemical substructures that are likely to show activity at D1. This algorithm was applied to an in silico screen of about 1,000,000 compounds representing random chemical libraries sold by 10 different vendors. From one library of 240,000 compounds, 400 were selected by the algorithm, purchased, and physically screened in the D1 assay. A hit rate of 8% was achieved, compared with hit rates of about 0.5%-1.0% (10-fold lower) for typical focused library screens and <0.1% (100-fold lower) for typical random library screens.

Parkinson 's Disease and Other Dopamine Agonist Applications

Parkinson's Disease is characterized by tremors and movement disorders that are the result of degeneration of brain cells that produce or release the neurotransmitter dopamine. Administering dopamine-like compounds such as levodopa (generic; multiple suppliers) can relieve symptoms of Parkinson's, and most drugs to treat this disorder (e.g., bromocryptine: Parlodel; Novartis) are dopamine receptor agonists. Drugs for Parkinson's Disease represent a current market of about $600 million, but with substantial upside in market potential given the inadequacies of current therapies. Although dopamine receptors are a clear target, there is still substantial uncertainty about which or how many dopamine receptor subtypes should be targeted for treating Parkinson's, with most previous attention being centered on the D2 subtype. Dopamine D1 agonists are also postulated as potential therapies for eating disorders. Parkinsonism symptoms can also be induced as a side effect of drugs, such as the antipsychotic drugs, that are antagonists of the dopamine D2 receptor. Therefore, understanding the molecular recognition patterns of drug candidates for the range of dopamine receptor subtype activities is of critical importance for both designing new dopamine subtype selective drugs and for controlling the side effects of drugs for other indications by selecting against activity at the dopamine receptors.

A number of drug candidates for Parkinson's Disease have exhibited adverse side effects, which in some cases has led to the cessation of development of the drug candidate. Such side effects can be due to interactions by the drug candidate with a number of other receptors or other molecular targets that mediate those side effects, in addition to those potential interactions with other dopamine receptor subtypes described above. The RSMDB and in silico screening methods have been used to identify potential drug candidates that exhibit the desired activity at the dopamine D1 receptor while failing to interact with up to six related molecular targets believed to be associated with the adverse effects of one drug candidate that had failed in development. Results of that in silico screening process, in which the chemical-target interaction RSMDB dataset and chemical substructural descriptors of the RSMDB compound set were used as a training set for computer-based screening of a large virtual compound library, are shown in the figure below.

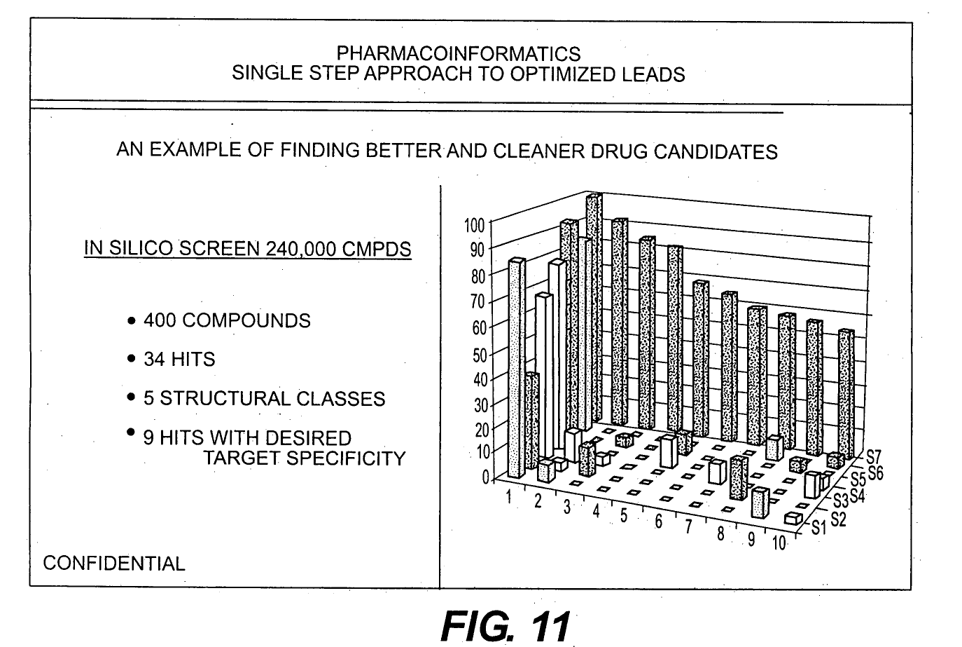

FIG. 11 shows a previous drug candidate that had failed development on the left, demonstrating its interactions with all molecular targets tested, and nine new compounds showing the desired positive interactions with the primary target (dopamine D1 receptor) but general lack of interactions with the six other targets believed to be mediators of the adverse side effects. The methods embodied allow for a one-step approach to optimizing the potency and selectivity of compounds for the desired molecular target and against undesired activity at other targets.

In addition to identifying compounds that are active as dopamine D1 agonists for the treatment of Parkinson's Disease, other target interactions that may contribute to the effcicacy of new drug candidates can be envisioned. In such cases it would be desirable to design or identify potential drug candidates that are simultaneously active at more than one target. It would be further desirable to identify compounds that are simultaneously active at more than one target and shown little or no activity at undesirable targets that mediate side effects or other adverse properties. The RSMDB and in silico screening methods described herein can also be used for this desired outcome.

Experimental Approaches

Step 1. Compile a chemical descriptor dataset targeting dopaamine D1 receptor activity as well as descriptor selectivity in seven (7) other “cocaine related” receptors using binding data mined from the RSMDB, and identify relevant and critical D1-selective chemical descriptors.

Step 2. Screen (via in silico methodologies) more than a million chemical structures available from suppliers of chemical compounds and select a subset of 1,000 compounds illuminated by the D-1 selective chemical descriptors identified in step 1.

Step 3. Screen (in vitro) the 1,000 selected compounds identified in Step 2 for activity at the dopamine D1 receptor using an in vitro radioligand binding assay. These seven in vitro binding assays include dopamine D2; serotonin 5HT2a; alpha-adrenergic 2a and 2b; beta-adrenergic 1 and 2; and norepinephrine transporter.

Example of Radio-Ligand Binding Assays

Donamine D₁ (Human Recombinant) BINDING ASSAY, [³H]-SCH 23390 as Radioligand

More information on the method of the aforementioned assay may be found in Jarvis et al., Molecular Cloning, Stable Expression and Desensitization of the Human Dopamine D_{1b}/D₅ Receptor. Jrnl. Receptor Research. 13(1-4): 573-590 (1993); and Billard et al., Characterization of the Binding of [³H]SCH 23390: a Selective D₁ Receptor Antagonist Ligand in Rat Striatum, Life Sciences, 35: 1885-1893 (1984) with modifications. Both of these references are herein incorporated by reference.

Tissue Preparation

Dopamine, D₁ recombinant receptor membranes expressed in HEK-293 cells are grown in the tissue culture facility. Membranes are stored in a −80° C. freezer until the day of the assay. Frozen pellets are thawed and diluted to 10 ug of protein/ml of assay buffer, so that the final concentration is 8 μg/ml, or 4 ug of protein per well. Alternatively, Cell Product vials are diluted directly to the assay buffer volume specified on the vial and homogenized without a centrifugation wash.

Binding Reaction

1. Each tube or well receives the following components:

50 ul of drug or vehicle

50 ul of [³H]-SCH 23390

400 ul receptor membrane preparation

2. Initiate the binding reaction with the addition of cell membranes and incubate at 25° C. for 60 minutes.

3. Terminate the binding reaction by rapid vacuum filtration of the assay tube contents onto presoaked (0.3% PEI for 3 hours) Whatman GF/B filters.

4. Rinse the assay tubes several times with ice-cold 50 mM NaCl.

5. The radioactivity trapped onto the filters is assessed using liquid scintillation counting.

Materials and Reagents

1. [³H]-SCH 23390 is diluted in 50 mM TRIS-HCl, pH 7.4, containing 10 mM MgCl₂, 5 mM KCl, 1 mM EDTA and 1.5 mM CaCl₂ to an initial concentration of 5.0 nM, such that the final radioligand concentration in the assay is 0.5 nM.

2. Non-specific binding is defined as that remaining in the presence of 2×10⁻⁷ M R(+)-SCH 23390.

3. The reference compound is R(+)-SCH 23390 run at the following final concentrations: 2×10⁻¹¹, 5×10⁻¹¹, 1×10⁻¹⁰, 2×10⁻¹⁰, 5×10⁻¹⁰, 1×10⁻⁹, 2×10⁻⁹, 5×10⁻⁹, 1×10⁻⁸, 2×10⁻⁸, 5×10⁻⁸, 1×10⁻⁷ M.

4. The positive control is R(+)-SCH 23390 run at final concentrations of 2×10⁻¹⁰, 2×10⁻⁹, and 2×10⁻⁸ M.

5. The K_{D} of [³H]-SCH23390 using the recombinant human D₁ receptor is 1.0 nM.

| | | | MW |
|---|---|---|---|
| BUFFERS | | | (g/mole) |
| Tissue Suspension | 50 mM Tris-HCl pH 7.4 | 6.05 g/L | |
| | 10 mM MgCl₂ | 0.95 g/L | 95.21 |
| | 1 mM EDTA | 0.38 g/L | 380.2 |
| | 5 mM KCl | 0.37 g/L | 74.55 |
| | 1.5 mM CaCl₂ | 0.17 g/L | 111 |
| Wash buffer: | 50 mM NaCl | | 58.45 |
| Filter Soak: | 0.3% PEI | | |

2. Examples of Cell Based Functional Assays (Agonist—Antagonist)

D1 Dopamine Agonist Assay (cAMP), Human Recombinant

More information on the method of the aforementioned assay may be found in Avalos, M. et al., Nonlinear analysis of partial dopamine agonist effects on cAMP in C6 glioma cells. J Pharmacol Toxicol Methods 2001 Jan-Feb; 45(1):17-37; and Monsma, F. J., et al., Molecular Cloning and Expression of a D₁ Dopamine Receptor Linked to Adenylyl Cyclase Activation Proc. Natl. Acad. Sci. USA. 1990 Sep. 1; 87 (17): 6723-6727. Both of these references are herein incorporated by reference.

Cell Preparation

HEK 293 cells expressing human dopamine D1 receptor were incubated in serum-free media overnight in microplates prior to cell treatment. 160 μL total culture volume per well is used for the agonist assay. Remove microplate plate from the incubator for initiation of assay procedure.

Agonist Assay

1. Drugs and controls are made in 4% DMSO (or lower % DMSO) whenever possible.

IBMX (3-isobutyl-1-methylxanthine) is made in serum free medium. All additions to the cells should be made as quickly as possible (within 5-15 minutes of the zero timepoint for the assay). IBMX should be added at 5 minutes before the zero timepoint.

2. Add 20 μL of 1 mM IBMX in serum free medium to each well, for a final concentration of 100 μM. Swirl gently to mix, and then allow to incubate for approximately 5 minutes (to allow drug and IBMX effects to equilibrate) at the assay temperature (37° C.).

3. Add 20 μL of the sample or reference compound dopamine (dopamine is the endogenous dopamine receptor agonist) to each well from a stock solution made at 10× the final concentration. The final concentration of DMSO will be 0.4%.

4. Add 20 μL of 100 μM forskolin in serum free medium to the positive control wells.

5. Incubate at 37° C. with the microplate lid on.

6. After 20 minutes incubation, carefully aspirate off the media. Then immediately add 200 μL/well of 0.1 M HCl. The cAMP to be measured by this assay is stable in HCl. Then seal the microplate with plastic film, and freeze the plate at −80° C. Freeze-thaw helps to permeabilize the cells. (Freeze-thaw may be repeated two more times.) Thaw and sonicate gently for approximately 2 minutes. Take care that liquid does not boil or otherwise evaporate fronr the plate. Take care also tnat liquid does not wick into the wells of me piate from the water bath. Sonication and warming need to occur evenly throughout the plate to prevent edge effects. Centrifuge the plate at 1500 rpm for 10 min. to remove debris. Use 10 μL of supernatant to perform the enzyme immunoassay (EIA) to measure cAMP (dilution factor is 20).

EIA Analysis

1. Use BioMol EIA kit (Format A cyclic AMP “Plus” Enzyme Immunoassay Kit, Catalog No. AK-215, BIOMOL Research Laboratories, Plymouth Meeting, Pa.).

2. Use 2000 pmol cAMP/mL standard provided in kit. Dilute 100 μL standard with 150 μL 0.1M HCl. Further dilute at 63 μL:187μL (i.e. 1:4), seven times, for a total of 8 standard tubes. Standard concentrations of cAMP are 800, 201.6, 50, 12.8, 3.23, 0.813, 0.2, and 0.05 pmol/ml. B₀ means 0 pmol/ml standard.

3. Follow the kit instructions of EIA assay procedure. The step 14 (adding 50 μl of Stop Solution) can be skipped. Only singlets of the eight cAMP standards and the four controls (blank, TA (total activity), NSD, B₀) are generally required. After antibody is added, plates may be incubated 2-3 hours at room temperature on a shaker, or overnight at 4° C. (preferred). Overnight incubation reduces background and enhances sensitivity by about three fold. Plates are washed 3× and pNPP (para-nitrophenylphosphate) substrate is added. Subsequent incubation time after pNPP addition may need to be adjusted according to room temperature (90-180 min.) or the samples can be placed in a 30° C. incubator for about 90 min. To maximize sensitivity, Bo should be in the 0.8 to 1.2 AU range. For non-overnight incubation, warm all reagents to room temperature before use.

4. Read enzyme reaction by measuring absorbance at 405 nm. One second/well reading time is suggested.

5. Analyze data according to instructions in kit. Calculate the average net Optical Density (OD) bound for each standard and sample by subtracting the average NSD OD from the average OD bound (sample—NSD). Then, calculate binding of each standard as a percentage of maximum binding (B₀). Plot Percent Bound (B/B₀) versus log of cAMP concentration for the standards. Samples should be in the linear range of the curve, with B/B₀ from 15 to 85%. With low cAMP levels, antibody incubation should be done overnight, at 4° C., to increase EIA sensitivity by about 3 fold. With high cAMP levels, 2-3 hour incubation at room temperature may be preferable. Sensitivity can be decreased by dilution of the 0.1M HCl cell supernatant, with a known amount of 0.1M HCl.

Materials and Reagents

1. Enzyme Immunoassay Kit: Format A cyclic AMP “Plus”, Catalog No. AK-205 or AK215, (BIOMOL Research Laboratories, Plymouth Meeting, Pa.) or equivalent.

2. 96-well plates: Costar polystyrene, flat bottom, low evaporation, sterile and tissue culture-treated with lids. (Costar catalog# 3370. VWR # 25381-056). For loosely attached HEK293 cells, tissue culture-treated plates were used (Costar catalog# 3585. VWR #29442-050).

3. The reference compounds are Dopamine (DA) (MW=189.6, Sigma catalog# H8502). Fresh Stock solution of DA (10 mM, 1E-2M) is made by adding 10 mg DA per 5.274 mL of 4% DMSO, Perform 7 1:10 dilutions starting at 1E-3M (1E-4M final) with 4% DMSO.

Final DA concentrations will be: 1E-10, 1E-9, 1E-8, 1E-7, 1E-6, 1E-5, IE-4 M. An eighth point with no DA is also run as part of the eight-point curve.

4. The EC50 for DA is approximately 53 nM.

5. IBMX (3-isobutyl-1-methylxanthine, MW=222.2, Sigma catalog# 17018) 1 mM solution is made fresh daily by adding 2.2 mg/10 mL serum free medium. The IBMX may need sonication (preferred) or brief boiling to become soluble.

6. Forskolin (MW=410.5, Sigma catalog #F6886). 10 mM stock solution is made in 100% DMSO and stored at −20° C. Daily, dilute 1:100 in serum free media to make a 100 μM working solution.

Dilution Tables for Making Standards 1-8:

| | 0.1 M HCl | Vol. Added | cAMP Cone. |
|---|---|---|---|
| Standard | Vol. (μL) | (μL) | (pmol/mL) |
| 1 | 150 | 100, Stock | 800 |
| 2 | 187 | 63, Std.1 | 201.6 |
| 3 | 187 | 63, Std.2 | 50 |
| 4 | 187 | 63, Std.3 | 12.8 |
| 5 | 187 | 63, Std.4 | 3.23 |
| 6 | 187 | 63, Std.5 | 0.813 |
| 7 | 187 | 63, Std.6 | 0.2 |
| 8 | 187 | 63, Std.7 | 0.05 |

D1 Dopaamine Antagonist Assay (cAMP), Human Recombinant

More information on the method of the aforementioned assay may be found in Avalos, M. et al., Nonlinear analysis of partial dopamine agonist effects on cAMP in C6 glioma cells, J Pharmacol Toxicol Methods 2001 Jan-Feb, 45(1): 17-37; and Monsma, F. J., et al., Molecular Cloning and Expression of a D1 Dopamine Receptor Linked to Adenylyl Cyclase Activation, Proc. Natl. Acad. Sci. USA. 1990 September 1; 87 (17): 6723-6727. Both of these references are herein incorporated by reference.

Cell Preparation

HEK 293 cells expressing human dopamine D1 receptor are incubated in serum-free media overnight before the cell treatment. 140 SL total culture volume is used per well for the antagonist assay. Remove plate from incubator prior to initiation of assay procedure.

Antagonist Assay

1. Drugs and controls are made in 4% DMSO (or lower % DMSO) whenever possible. IBMX (3-isobutyl-1-methylxanthine) is made in serum free medium. All additions to the cells should be made as quickly as possible (within 5-15 minutes of the zero timepoint for the assay). IBMX should be added at 5 minutes before the zero timepoint.

2. Add 20 μL per well of 1 mM IBMX in serum free medium, for a final concentration of 100 μM. Swirl gently to mix, and then incubate for approximately 5 minutes (to allow drug and IBMX effects to equilibrate) at assay temperature (37° C.).

3. Add 20 μL of the sample or reference compound (SCH23390, D1 specific antagonist), at 10× the final concentration for 5 min. Then, add 20 μL of 10 μM D1 agonist dopamine (i.e. 1 μM final concentration of dopamine) to each well.

4. Add separate 20 μL of 100 uM forskolin in serum free medium to positive control wells.

5. Incubate at 37° C. with the microplate lid on.

6. After 20 minutes incubation, aspirate off the media. Then immediately add 200 μL/well of 0.1 M HCl. The cAMP to be measured by the assay is stable in HCl. Then seal microplate with plastic film, and freeze plate at −80° C. Freeze-thaw helps to permeabilize the cells. (Freeze-thaw may be repeated two more times.) Thaw and sonicate gently for approximately 2 minutes. Take care that liquid does not boil or otherwise evaporate from the wells of the plate. Take care also that liquid does not wick into the wells from the water bath. Sonication and warming need to occur evenly throughout the plate to prevent edge effects. Centrifuge the plate at 1500 rpm for 10 min. to remove debris. Use 10 μL of supernatant to perform the enzyme immunoassay (EIA) to measure cAMP (dilution factor is 20).

EIA Analysis

1. Use BioMol EIA kit (Format A cyclic AMP “Plus” Enzyme Immunoassay Kit, Catalog No. AK-215, BIOMOL Research Laboratories, Plymouth Meeting, Pa.).

2. Use 2000 pmol cAMP/mL standard provided in kit. Dilute 100 μL standard with 150 μL 0.1M HCl. Further dilute in 63 μL:187 μL (i.e. 1:4) ratio, seven times, for a total of 8 standard tubes. Standard concentrations of cAMP are 800, 201.6, 50, 12.8, 3.23, 0.813, 0.2, and 0.05 pmol/ml. B₀ means 0 pmol/mL standard.

3. Follow the kit instructions of EIA assay procedure. The step 14 (adding 50 μl of Stop Solution) can be skipped. Only singlets of the eight cAMP standards and the four controls (blank, TA (total activity), NSD, B₀) are generally required. After antibody is added, plates may be incubated 2-3 hours at room temperature on a shaker, or overnight at 4° C. (preferred). Overnight incubation reduces background and enhances sensitivity by about three fold. Plates are washed 3× and pNpp substrate is added. Subsequent incubation time after pNpp addition may need to be adjusted according to room temperature (90-180 min.) or the samples may be placed in a 30° C. incubator for about 90 min. To maximize sensitivity, B₀ should be in the 0.8 to 1.2 AU range. For non-overnight incubation, warm all reagents to room temperature before use.

4. Read enzyme reaction by measuring absorbance at 405 nm. One second/well reading time is suggested.

5. Analyze data according to instructions in kit. Calculate the average net Optical Density (OD) bound for each standard and sample by subtracting the average NSD OD from the average OD bound (sample—NSD). Then, calculate binding of each standard as a percentage of maximum binding (B₀). Plot Percent Bound (B/B₀) versus log of cAMP concentration for the standards. Samples should be in the linear range of the curve, with B/B₀ from 15 to 85%. With low cAMP levels, antibody incubation should be done overnight, at 4° C., to increase EIA sensitivity by about 3 fold. With high cAMP levels, 2-3 hour incubation at room temperature may be preferable. Sensitivity can be decreased by dilution of the 0.1M HCl cell supernatant, with a known amount of 0.1M HCl.

Materials and Reagents

1. Enzyme Immunoassay Kit: Format A cyclic AMP “Plus”, Catalog No. AK-205 or AK215, (BIOMOL Research Laboratories, Plymouth Meeting, Pa.) or equivalent.

2. 96-well plates: Costar polystyrene, flat bottom, low evaporation, sterile and tissue culture-treated with lids. (Costar catalog# 3370. VWR # 25381-056). For loosely attached HEK293 cells, tissue culture-treated plates were used (Costar catalog# 3585. VWR #29442-050).

3. The reference compound is SCH23390 (MW=324.1, RBI catalog# D054). Run control wells in triplicate containing only a final concentration of IE-6 M dopamine (DA). Make sufficient 10 μM DA (1 μM final) and add 20 μL to each antagonist well (except DA controls).

Use a fresh aliquot daily, or avoid rethawing of frozen aliquots. Perform 7 1:10 dilutions, starting at 1000 uM (1E-4M final), using 4% DMSO, serum-free media. Final SCH23390 dilutions will be 1E-10, 1E-9, 1E-8, 1E-7, 1E-6, 1E-5, 1E-4 M. An eighth point with no SCH23390 is also run as part of the eight-point curve.

4. The IC50 for SCH23390 is 4.3 nM.

5. IBMX (3-isobutyl-1-methylxanthine, MW=222.2, Sigma catalog# 17018) 1 mM solution is made fresh daily. The IBMX may need sonication (preferred) or brief boiling to become soluble.

6. Forskolin (MW=410.5, Sigma catalog #F6886). 10 mM stock solution is made in 100% DMSO and stored at −20° C. Daily, dilute 1:100 in serum free to make a 100,M working solution.

Dilution Tables for Making Standards 1-8:

| | 0.1 M HCl | Vol. Added | cAMP Conc. |
|---|---|---|---|
| Standard | Vol. (μL) | (μL) | (pmol/mL) |
| 1 | 150 | 100, Stock | 800 |
| 2 | 187 | 63, Std.1 | 201.6 |
| 3 | 187 | 63, Std.2 | 50 |
| 4 | 187 | 63, Std.3 | 12.8 |
| 5 | 187 | 63, Std.4 | 3.23 |
| 6 | 87 | 63, Std.5 | 0.813 |
| 7 | 187 | 63, Std.6 | 0.2 |
| 8 | 187 | 63, Std.7 | 0.05 |

Results

The following discussion (summarized in Table 5) perhaps uses one of the best examples and precedents to illustrate validity of the. proposed approach. In a study that was unrelated to this proposal, the goal was to identify compounds selectively reactive with only one (D1) of seven GPCR receptors, whereas all 7 receptors demonstrated a high degree of sequence homology. A full-rank training matrix of 1,573 compound x 7 biological targets was used to build 7 individual partitioning trees; each “tree” was related to an individual target; all trees were built with the same compound set, unprejudiced towards any of the seven targets within the array.

TABLE 5
Summary of GPCR screening result using parallel triage methodology

| | | | | | | Number of |
|---|---|---|---|---|---|---|
| | | | | | Hit Rate | Selectives (5 |
| | Target | Target | Number of Hits | Hit Rate | Imporvements | folds over |
| Target ID | Similarities (%) | Identities (%) | (50% cut off) | (%) | (over 0.1%) | others) |
| T1 | 55 | 30 | 9 | 2.25 | 22.5 | 0 |
| T2 | 69 | 49 | 8 | 2 | 20 | 0 |
| T3 | 60 | 32 | 8 | 2 | 20 | 1 |
| T4 | 62 | 48 | 7 | 1.75 | 17.5 | 0 |
| T5 | 55 | 38 | 16 | 4 | 40 | 0 |
| T6 | 63 | 42 | 24 | 6 | 60 | 4 |
| T7 | 100 | 100 | 34 | 8.5 | 85 | 9 |

It has long been known that similar biological targets are likely to have similar chemical activity profiles; and that similar chemicals are likely to have similar biological profiles. Such experience has long been the guiding principle of “focused pharmaceutical screening”. From a library of 250,000 compounds and using the “positive leaves” of the D1 partitioning trees, we compiled a “long” list of compounds (40,000) that are statistically likely to be reactive with D1 due to the presence of the “positive” descriptors. For target relatedness (homologies between them), this list of compounds will likely be reactive within the array. However, this “long” list was then further “trimmed” with the “negatives leaves” of six other “trees” related to the aforementioned array of biological targets. The “trimming” process is to use the “negative” nodes to select compounds from the list of 40,000-compounds that already exhibited (in silico) likelihood of D1 activity. Each “trimming” step afforded a smaller subset that is likely to be active against D1 and less likely to be active against another for the list was “picked” using positive leaves of D1 and negative leaves of another tree. The final subset, much smaller than the original, contains molecules that are having positive chemical descriptors for D1 and negative descriptors for all six other targets. The list was then further “trimmed” or examined using “Lipinsky rule of five” for drug likeness and diversity assessments to afford a 406-compound library, 1% of the original long list, 0.16% of the original library of 250,000 compounds.

Table 5 summarizes the result of screening. The entire collection, 406 compounds was screened against the entire target array of seven targets at 10⁻⁵ M. Against D1, 34 compounds, representing >5 distinctly different structure classes, exhibited more than 50% inhibitory activity, constituted a hit rate of 8.5% and demonstrated a 85-fold increase in hit rate (or productivity) as compared to the conventional screening of random chemical library (hit rate of 0.1%). On average, overall hit rates against all 7 targets are about 30%. These resuits approximate our expectations.

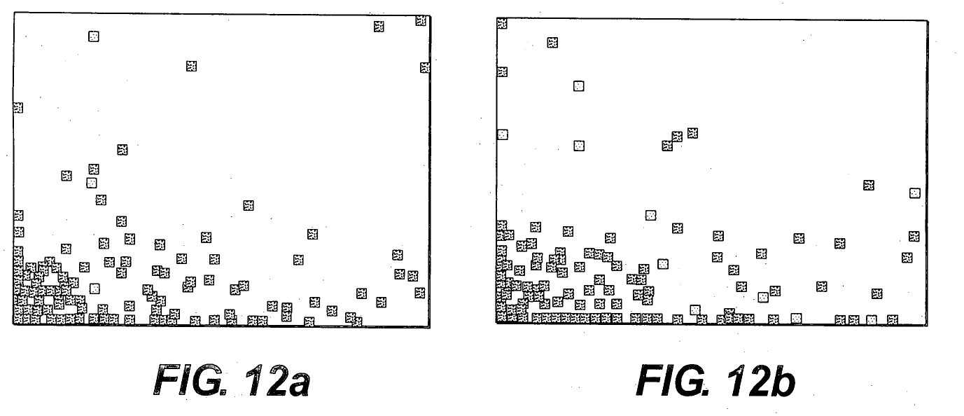

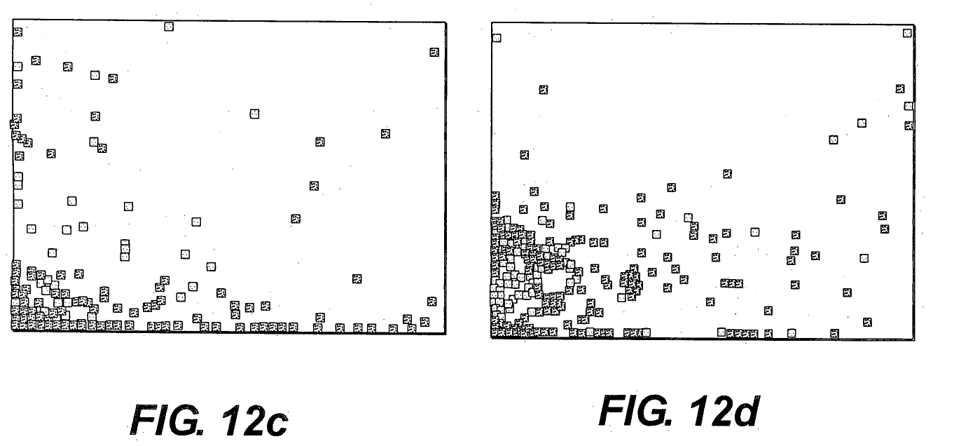

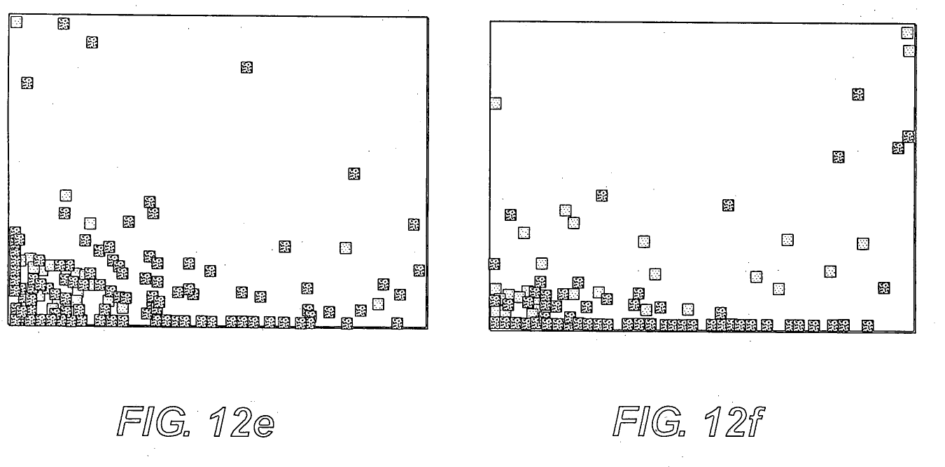

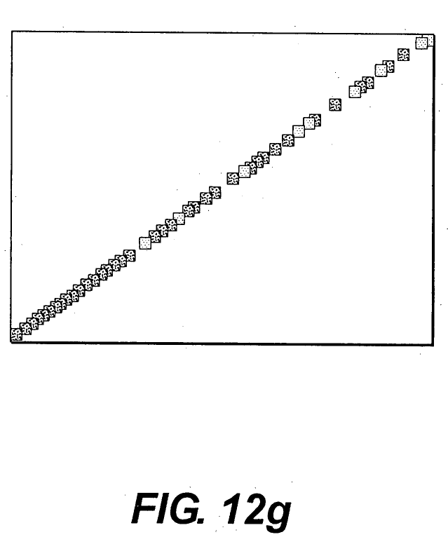

The more important concerns, in light to this proposal, are the selectivity profiles of those found to be active against D1. FIG. 12 is the “overall landscape” of the activity profiles of the 406×7 full matrix illustrated in a collage of scatter-plots. This is an activity profile of 406 compounds screened against 7 GPCR targets. The horizontal axis in the each graph represents target, D1, and the scales represent inhibitory activities of the 406 tested compounds. Likewise, the vertical axis represents 7 individual GPCR targets in the chosen array; as well as the inhibitory activities of the 406 compounds. Note in graph “g” that both axis represent D1. In each scatter-plot, the axes represents different receptor activity, and the scale of the axis represent percent inhibition obtained from specific receptor radio-ligand binding assays. In scatter-plot “g”, both axis are representing D1, hence the data points are distributed along the 45° angle of the plot. In other six scatter-plots, a-f, the X-axis is D1 whereas the Y-axis' represent these other targets in the array. As shown in each pair-wise comparison (using this type of scatter-plot), there is an apparent “gravitational pull” of data along the X-axis, which indicate that the entire library is biased for a selective D 1 activity.

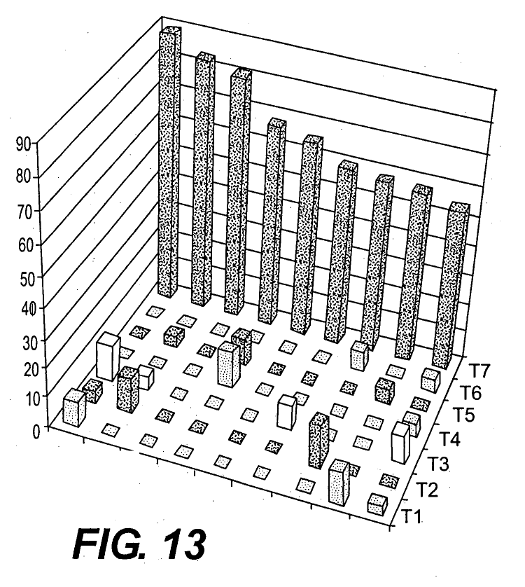

More impressively, 9 compounds showed nearly specificities with D1 (activities are 5 folds more reactive with D1 than with any others of the same array). The reactivity profiles of the 9 compounds are summarized in FIG. 13, which demonstrates that 9 compounds showed nearly specific activity with D1 for their activities are 5 folds more reactive with D1 than with any others of the same array. In conclusion, these examples have demonstrated the possibility of “translating” the “probability differential” to selected reactivity or even target specificity in a given set of GPCR targets.

### Example V

### Compounds Active at Two or More Therapeutic Targets and Inactive at a Multiplicity of Potential Side Effect Targets for Treating Parkinson's Disease—In Silico Screening Methods for New Compound Discovery

I. Rational of Target Composition and Technical Background—Experts estimate that 1 percent of the U.S. population over 60 years old will fall prey to debilitating Parkinson's disease. About 1 million Americans now suffer from the disease. With the increase of the average life span, the problem is getting worse for more people experiencing the disease, and the patients will deal with the disease for a long time. In addition, because of the increasing population of Parkinson's disease, the costs of long term care and medical care will be dramatically increasing.

The root of Parkinson's disease, marked by the degeneration of dopaminergic neurons in the substantia nigra with onset of motor symptoms, represents one of the most challenging brain degenerative diseases to the pharmaceutical community. Medications are limited thus far to symptomatic therapy using L-Dopa and/or dopaminergic receptor agonists like pergolide, ropinirole and pramipexols.

Dopamine replacement therapy (with L-Dopa) is highly effective in the early stage of Parkinson's disease. With time, the efficacy of L-Dopa declines and effective duration become shorter and unpredictable, in fact, 20 to 30% patients treat with L-Dopa develop abnormal movements collectively called dyskinesia. Both L-Dopa and dopamine agonists (sometime used in combination) can induce psychosis. For instance, Pergolide (Permax), a dopaminergic receptor agonist introduce in 1989 is listed with the following side-effects: anxiety, restlessness, confusion, double vision, fainting spells, hallucinations, headache, mental changes, palpitations and uncontrollable movements of the arms, face, hands, head, mouth, shoulders, or upper body.

Clearly, better drugs, efficacious with prolonged and repeated applications and with much less debilitating side effects are needed.

With the success of the KW-6002-US-02 Phase Ia trial (see Kanda et al., “Actions of Adenosine Antagonists in Primate Model of Parkinson's Disease,” Adenosine Receptors and Parkinson's Disease, Academic Press, p: 211-227, 2000, which is herein incorporated by reference), the A2A antagonist KW-6002 (see Hubble et al., “A Novel Adensosine Antagonist (KW-6002) as a Treatment for Advanced Parkinson's Disease with Motor Complications,” Neurology 2002, 58 (supplement 7), S21.001, A162, which is herein incorporated by reference) was validated and established that the adenosine A_{2A} receptor is a novel target. Selective A_{2A} antagonists, such as KW-6002, could be the next generation of new therapy to stamp out some of the pain and suffering of the Parkinson's disease suffers.

Notably, in one of the early reports, the combined use of KW-6002 with L-Dopa or with selective dopamine agonists (D1 or D2) potentiate the antiparkinsonian effect but does not induce dyskinesia in MPTP-treated monkeys (see Kanda et al.). The same potentiation is observed lately in the human trials (see Sherzai, et al., “Adenosine A2a Antagonist Treatment of Parkinson's Disease,” Meurology 2002, 58 (supplement 7), S21.001, A162.P06.104, A467, which is herein incorporated by reference).

There is a market need and a demand for new antiparkinson therapeutics. The demand for new antiparkinson therapeutics is generated by the deleterious side effects of current regimens. Table 1 presents a partial activity profile of Pergolide (Permax), a dopamine agonist registered to be used in antiparkinson therapy. Side effects of this type of drug are well known to the patient populations. Drug induced prolonged psychotic episodes prevent significant patient populations from continuing treatment due primarily to the combination of pathology and drug effects. Side effect profiles of KW-6002 are currently unavailable so that tolerance to the drug and induced psychological impact under prolonged application are unknown.

The clinical uses of dopamine receptor agonists and adenosine receptor antagonists provide the proof of principal of the validity of theses therapeutic targets, both individually and together. The mutual complementation of these receptors with limited side effects indicates a beneficial receptor synergism and activity, hence providing the justification for seeking small molecules with the desired receptor (A_{2A}) antagonist activity or antagonist (A_{2A}) and concurrent agonist activity (D1 or D2). Compounds with potent and selective activity at these receptors and the “correct” physical chemical properties will likely result in leads with potential therapeutic activity. Identifying (from a population of chemical entities) a single chemical entity with potent and concurrent activities at more than one receptor as well as selectivity within an extended family of related and unrelated receptors is the essence of finding better drugs. Compounds that are efficacious with minimum side effects is the focus of this project.

The objective of this example is to seek novel chemical entities acting as selective A_{2A} antagonists, or chemical entities acting as selective A_{2A} antagonists and concurrently as selective D1 agonists or selective D2 agonists. These leads will be further developed initially as research tools, and then a panel of leads and candidates will be selected as a new generation of antiparkinson therapeutics. Discovering an efficacious drug is a difficult task. Facing this challenge, this example instituted two key technical innovations.

First, this example takes multiple biological targets into considerations simultaneously and early in the discovery phase to address issues related to efficacy, side effects and drug safety. The selection of pharmacological target array, within which the issues of receptor selective activity is addressed, is closely related to the concerns of in vivo side effects and associated in vitro activity proflcs. For cxarlmple, in this proposal a population of compounds active against A_{2A}, or compounds active against A_{2A} and D1 simultaneously, likewise against A_{2A} and D2 is being sought. Within the same population of compounds, a lack of prominent activity at other related receptors such as A_{1A}, and selectivity within the family of dopamine receptors, is also being sought. Additionally and perhaps more importantly, again within the same population of compounds, a lack of prominent activity at the receptors relevant for CNS or cardiovascular side effects, is being sought. The target selection regarding the unintended effects included selected adrenoceptors, serotonergic receptors, muscarinic receptors and monoamine transporters.

II. Experimental Approaches—Listed by Steps

Step 1. Identify chemical descriptors associated with biological activities observed at the adenosine receptor (A_{2A}), and at the dopamine receptors D1 and D2, and then identify chemical descriptors devoid of other selected receptor activities.

Step 2. Use the identified chemical descriptors to identify compounds in silico (from a collection of libraries >1.1 million compounds) that are potentially active against A_{2A} or potentially and concurrently active at A_{2A} and D1 or at A_{2A} and D2. Identify which of these compounds are potentially and concurrently inactive against adenosine A_{1A}, 5HT_{1A}, 5HT₃, norepinephrine transporter (NET), dopamine transporter (DAT) and serotonin transporter (SERT), adrenergic receptors α_{1A}, α_{1B}, α_{2B}, M₁, M₂ and M₃. This activity/inactivity fingerprint analysis is based on the statistical data interrogation of Step 1.

Step 3. Use a computational program to identify compounds (resulting from Step 2) defined by Lipinsky's “rule of five” for drug-likeness.

Step 4. Compile and acquire 1,500 compounds identified by the applied selection criteria from different vendors.

Step 5. Screen the acquired compound collection (1,500 compounds) for activity against A_{2A} and D1 and D2 using radioligand binding assays at 10⁻⁵M concentration and identify those compounds (hits) active against A_{2A} and/or at both receptors, A_{2A} and D1, and/or both A_{2A} and D2;

Step 6. Screen these “hits” (identified in Step 5) using radioligand binding assays at same concentration as Step 5 against the a A_{1A}, 5HT_{1A}, 5HT₃, norepinephrine transporter (NET), dopamine transporter (DAT) and serotonin transporter (SERT), adrenergic receptors α_{1A}, α_{1B}, α_{2B}, β1, β2, and β3.

III. Results—

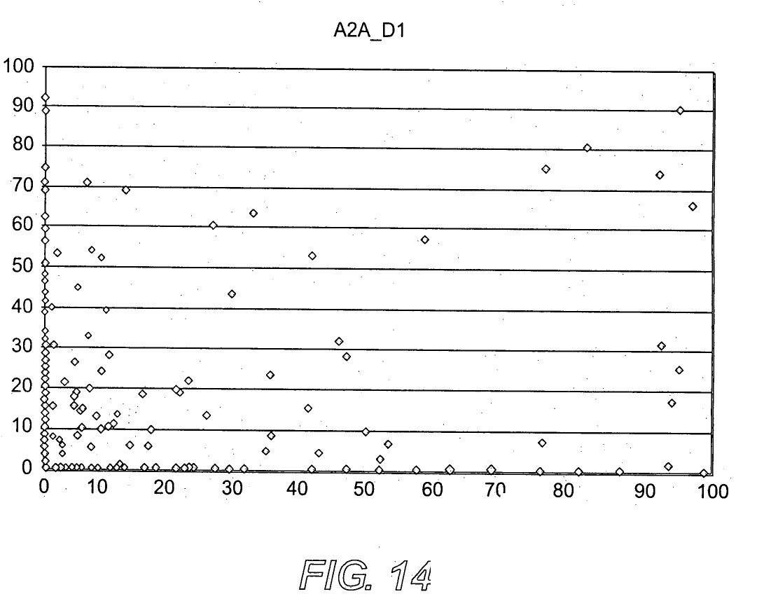

This example demonstrates that using the existing database, the platform enables discovery research to find compounds with a designated profile of activities-inactivities. In this example the objective is seeking compounds against a pair of GPCRs, A_{2A} (antagonist)-D1 (agonist). The focus of the project is seeking compounds that are potentially useful in treating Parkinson's disease. FIG. 14 gives a preliminary result of screening about 600 compounds against the pair of GPCRs. This is an initial data set obtained from the testing a panel of 600 compounds against dopamine D1 (X) and adenosine 2A (Y) activity. The compounds were selected based on dopamine D1 agonist and A2a antagonist models. The library is comprised of compounds biased for A2a antagonist, D1 agonist and compounds with concurrent activities of D1-A2a. The data points at the upper right hand corner indicated a few compounds demonstrating potent and selective activity with both receptors. Comparing this “yield” with a convention HTS (1/10⁶ probability), the improvement is significant. Please also note, similar to this proposal, that is, this data set presented herein also include those compounds selected only dopamine and adenosine receptor activity only. Hence those data points along both axis.

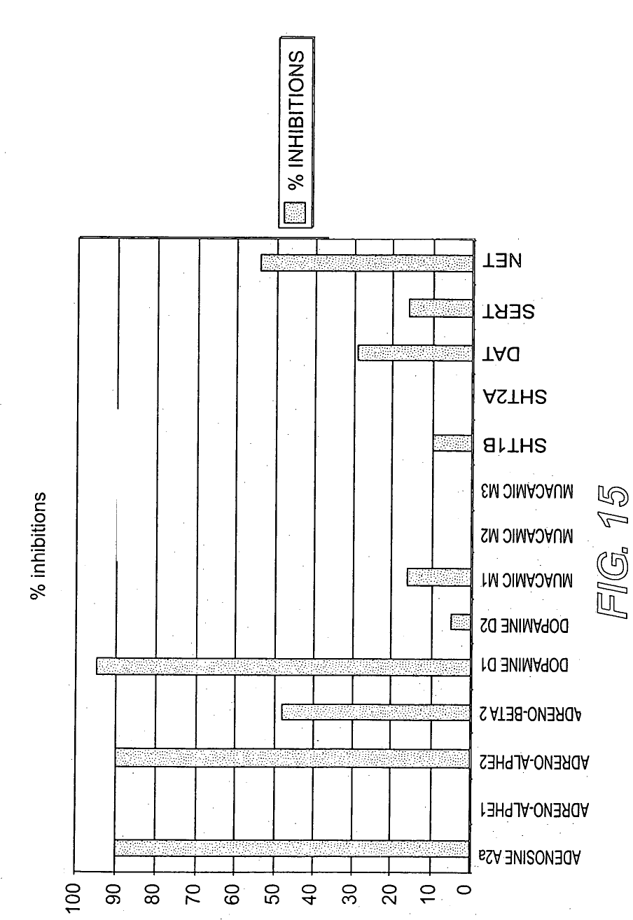

The compounds that are shown to have a dual modulator activities, also have shown a reasonable profiles of receptor selectivities. In FIG. 15, a partial profile shows that the lead compounds identified using the described method are selective. This is the activity profile of a lead compound demonstrating concurrent activity with D1 and Adenosine A2a. Most of the other activity apparently are eliminated or diminished. However, the activity at adreno-alpha1 2 is some what unexpected.

### Example 6

### Compounds Active at Two or More Therapeutic Targets for Treating Drug Dependency or Overdose, Anxiety or Insomnia—Direct Database Interrogation and In Silico Screening Methods for New Compound Discovery

Barbiturate Dependency/Overdose—Benzodiazepine Receptor Agents—Barbiturates (“sleeping pills”) were introduced in 1903 as sedative-hypnotic drugs and, while generally replaced by new classes of sedative-hypnotic drugs such as the benzodiazepines and others, are still widely used—and abused. Patterns of abuse include people with emotional disorders using these pills to escape reality and/or people using the pills for a short-term altered mental state and lowered inhibition, much like the abuse of alcohol. Attempts to break the dependence or addiction often leads to severe and unpleasant withdrawal symptoms. The benzodiazepine class of drugs that replaced barbiturates as sedative-hypnotics also is prone to abuse. For example, flunitrazepam (Rohypnol, Roche) has gained notoriety as the “date rape” drug. A need exists for a safe and effective drug to combat barbiturate dependence, manage withdrawal, and to treat barbiturate or benzodiazepine overdose or acute poisoning.

The mode of action for both barbiturates and benzodiazepines is mediated through a receptor called the GABA A—benzodiazepine central receptor. There are a number of different subtypes and different sites of action of compounds on GABA receptors. Advances in genomics have demonstrated even more complexity for the GABA receptors with different subunits coming together to form different functional receptor units. We have developed a number of GABA receptor subtype assays, which are included in the RSMDB.

Compounds that inhibit the interaction between benzodiazepines or barbiturates and the GABA A benzodiazepine receptor are candidates for such an anti-barbiturate abuse agent. Through the RSMDB, compounds are being searched for that act as antagonists at the GABA-A benzodiazepine receptor as potential medications for barbiturate dependency and acute overdose. In addition this program is directed toward finding GABA A-benzodiazepine agonists that may have potential as sedative-hypnotic (anti-anxiety) drugs with more significant market potential.

Anxiety (Sedarive-Hypnotic) Drugs—Sedative-hypnotic drugs are used for causing sedation (treating anxiety) and encouraging or inducing sleep. Other related indications include anesthesia, anticonvulsants, muscle relaxants, and respiratory function control. Sedative-hypnotics are among the most widely prescribed drugs worldwide, with estimated sales of $7.8 billion.

The most important chemical class of sedative hypnotic drugs has been the benzodiazepines (such as alprazolam: Xanax, Pharmacia-Upjohn; and triazolam: Halcion, Pharmacia-Upjohn), which have as their primary mode of action agonism of the GABA-A, benzodiazepine receptor. Each of these chemicals has the same basic chemical structure, or pharmacophore, and all share some common side effects and modes of action. Newer drugs in this chemical class have been designed for greater selectivity for the intended benzodiazepine target, which in turn results in fewer side effects and gains in market share. This chemical class of drugs remains, however, with significant interactions with other receptors that may mediate undesirable side effects. Substantial market opportunities exist for unique chemical classes that might provide equal or greater efficacy with fewer side effects. Several newer chemical compound classes have been introduced for treatment of anxiety and sleep disorders. One of these is buspirone (BuSpar, BristolMyersSquibb), which does not work through the benzodiazepine receptor but instead is an agonist at the serotonin 5HT1A receptor. It lacks some of the broader effects of benzodiazepines such as sedation, which could be considered an unwanted side effect when just treatment of anxiety is desired. Another new chemical group (zolpidem: Ambien, Pharmacia-Upjohn; and zaleplon: Sonata, American Home Products) binds selectively to a subtype (omega 1) of the benzodiazepine central receptor. These improved drugs appear to have lower risk of side effects compared with benzodiazepine drugs and have gained significant market share.

Through the RSMDB, a compound (NBC-52100) has been identified that is highly active at the GABA-A benzodiazepine receptor but has an entirely different type of chemical structure, or pharmacophore, compared with agents currently on the market. Furthermore, NBC-52100 shows activity at the 5HT1A receptor but exhibits virtually no other receptor interactions among the targets in the RSMDB, suggesting it may have significantly reduced side effects. This compound is a known chemical marketed for non-pharmaceutical applications and has a proven safety profile in animal studies. NBC-52100 demonstrates in vivo activity in rodents and is entering preclinical testing. The pharmacoinformatics platform and in silico screening methods can also be used to identify additional compounds containing the same pharmacophore for further development as second-generation drug candidates.

This embodiment relates to the treatment of conditions in mammals by administration of a composition that interacts as an agonist at the GABA-Albenzodiazepine receptor and at the 5HT1A receptor and in particular to such treatments which involve the administration of carotenoid synthesis inhibiting herbicidal agents.

This embodiment identifies a class of compounds, represented by fluridone (NBC-52100), which is highly active at the GABA-AIbenzodiazepine receptor but has an entirely novel type of chemical structure or pharmacophore, a pyridinone, when compared with currently known agents. Furthermore, fluridone shows some activity at the 5HT1A receptor but exhibits virtually no other significant receptor interactions, suggesting it may have significantly reduced side effects.

Fluridone is a known chemical approved for agrochemical use as an herbicide with a known biochemical mechanism of herbicidal activity. In plants fluridone acts as an inhibitor of an essential enzyme, phytoene desaturase, which catalyzes a critical step in the biosynthesis of carotene and carotenoid pigments. Plants treated with fluridone cannot biosynthesize carotenoids and consequently become bleached and die when exposed to sunlight. Fluridone has a proven safety profile in animal studies.

In one embodiment a composition and a method for treating a condition in a mammal treatable by the administration of a GABA-Albenzodiazepine receptor agonist or partial agonist, which includes administering to the manmmal a therapeutically effective amount of a carotenoid synthesis inhibitory herbicidal agent. Such agents include, for example, pyridinone compounds, for example, encompassed by the pyridinone compounds presented in U.S. Pat. No. 4,152,136, which is hereby incorporated by reference herein by reference in its entirety.

In a particularly preferred form, the pyridinone compound is fluridone: 1-methyl-3-phenyl-5-(α, α, α-trifluoro-m-tolyl)-4-pyridone.

A profile of the pharmacological activity of fluridone in 98 pharmacologically relevant receptors and enzymes in in vitro assays were determined. Please consult Table 6 for a tabulation of Fluridone's activity in a panel of 98 receptor-binding and enzyme assays.

Fiuridone demonstrates significant binding activity only in the GABA-A/Benzodiazepine Central receptor assay. The activity of fluridone on subtypes of the GABA-A/benzodiazepine receptor was determined in in vitro assays.

Activity of Fluridone in the GABAA-BZ subtypes in in vitro assays.

$$\begin{matrix} & \underline{alpha1 alpha5 alpha6} \\ Ki , nM370 & 344 \; 114 > 100 , 000 \end{matrix}$$

Fluridone does not recognize the GABA_{A}-α6 benzodiazepine site. By extension, it probably does not recognize diazepam insensitive sites, which include α4 and α6. Conversely, it recognizes α1 and α5 with moderate affinity, and probably recognizes other diazepam sensitive sites, including α2 and α3.

The in vivo results are consistent with GABA-A α1 interactions, that is, sedative hypnotic effects since the agent is active at the alpha1 site with a Ki=3.7×10⁻⁷ Molar.

The in vivo effects of Fluridone were examined in a mouse model. Fluridone was injected i.p. in order to determine its effects on the animal. To further characterize the effects of the agent, Fluridone was injected prior to an injection of bicuculline, a drug that is known to be a GABA-A antagonist and known to induce seizures and death when injected at elevated dosages. The Fluridone/bicuculline combination constituted an in vivo GABA-A agonism/antagonism assay.

After administering a substantial dose of Fluridone (250 mg/kg), the tails of the test mice stood straight up and the mice fell on their sides. This appears to be an opiate-like effect. The treated mice recovered from the initial opiate-like effect within one minute and regained their normal stance and tail display. The mice became sedated but breathing remained normal and the heart rate decreased somewhat. All treated mice displayed no evidence of seizure and all mice survived the treatment. Recovery from the treatment occurred over the course of several hours. These mice were observed over the next two days and displayed no visual effects of the Fluridone treatment over that period.

Injection of high doses of bicuculline (5 mg/kg) induced immediate seizures in mice. The injected mice all displayed tail curvature and their bodies become rigid. The mice died shortly after seizing. Death rate was 100% within ten to twenty second after bicuculline injection.

Injection of Fluridone (250 mg/kg) one hour prior to injection of bicuculline (5 mg/kg) clearly demonstrated that Fluridone pretreatment protects mice from the effects of bicuculline. In this situation, approximately 50% of the treated mice experienced mild seizures and 100% of the mice survived the treatment. These mice were observed over the next two days and displayed no visual effects of the Fluridone/bicuculline treatment over that period.

Fluridone is able to protect against bicuculline-induced GABA-A antagonism-related seizures and death in the mouse model. Consequently, Fluridone acts as a GABA-A agonist in vivo.

Table 6 below tabulates the activity of Fluridone when the agent was tested in 98 different designated receptor-binding and enzyme activity assays. For the initial inhibition column, the activity of Fluridone was determined at a 10 uM concentration in each assay. If some activity was present at 10 uM, then further analysis was performed and is presented in the verify_{—}3 column. Specifically, Fluridone's activity was determined at 10 uM, 100 nM and 1 nM concentrations and again presented as a % inhibition at 10 uM value. A Ki value for Fluridone was determined for the GABA-A receptor.

TABLE 6

| | | Inhibition | | |
|---|---|---|---|---|
| receptor_ID | name | 10 uM | verify_3 | Ki value |
| 1 | Adenosine Transporter | 22.93% | | |
| 2 | Adenosine, A1 | −29.89% | | |
| 3 | Adenosine, A2A | 31.61% | 36.02% | |
| 5 | Adrenergic, Alpha 1A | 19.32% | | |
| 6 | Adrenergic, Alpha 1B | 9.13% | | |
| 7 | Adrenergic, Alpha 2A | −2.61% | | |
| 8 | Adrenergic, Alpha 2B | 9.57% | | |
| 9 | Adrenergic, Alpha 2C | −3.60% | | |
| 10 | Adrenergic, Beta 1 | −15.27% | | |
| 11 | Adrenergic, Beta 2 | −3.08% | | |
| 12 | Bradykinin, BK2 | 23.51% | | |
| 13 | Calcium Channel, | 19.32% | | |
| | Type L (DHP Site) | | | |
| 14 | Calcium Channel, | −4.69% | | |
| | Type N | | | |
| 15 | Dopamine Transporter | 12.41% | | |
| 16 | Dopamine, D1 | −8.43% | | |
| 17 | Dopamine, D2s | 15.30% | | |
| 18 | Dopamine, D3 | 11.88% | | |
| 19 | Dopamine, D4.4 | 29.11% | | |
| 20 | Dopamine, D5 | 8.40% | | |
| 21 | GABA A, Agonist Site | 25.20% | | |
| 22 | GABA, | 104.70% | 96.73% | 2.9E−7 |
| | Benzodiazepine, α1 | | | |
| 23 | GABA, Chloride, | 53.09% | 31.38% | |
| | TBOB Site | | | |
| 24 | GABA-B | 14.56% | | |
| 25 | Glucocorticoid | 2.23% | | |
| 26 | Glutamate, AMPA | 12.15% | | |
| | Site | | | |
| 27 | Glutamate, Kainate | −4.07% | | |
| | Site | | | |
| 28 | Glutamate, MK-801 | 1.23% | | |
| | Site | | | |
| 29 | Glutamate, NMDA | −28.91% | | |
| | Agonist Site | | | |
| 31 | Glutamate, NMDA, | 3.83% | | |
| | Phencyclidine Site | | | |
| 32 | Glutamate, NMDA, | −4.53% | | |
| | Glycine (Stry-insen.) | | | |
| 33 | Glycine, Strychnine- | −3.07% | | |
| | Sensitive | | | |
| 34 | Histamine, H1 | −1.76% | | |
| 35 | Histamine, H3 | 13.22% | | |
| 36 | Leukotriene, LTB4 | 19.25% | | |
| 37 | Leukotriene, LTD4 | −0.69% | | |
| 38 | Muscarinic, M1 | 12.32% | | |
| 39 | Muscarinic, M2 | 6.52% | | |
| 40 | Muscarinic, M3 | 7.77% | | |
| 41 | Muscarinic, M4 | 15.71% | | |
| 42 | Muscarinic, M5 | 5.70% | | |
| 44 | Neurokinin, NK1 | −1.41% | | |
| 45 | Neuropeptide, NPY2 | −7.66% | | |
| 46 | Nicotinic, | −6.04% | | |
| | (a-bungaro-toxin | | | |
| | insensitive) | | | |
| 48 | Norepinephrine | 8.90% | | |
| | Transporter | | | |
| 49 | Opiate, Delta | 21.50% | | |
| 50 | Opiate, Kappa | −13.89% | | |
| 51 | Opiate, Mu | 7.75% | | |
| 52 | Potassium Channel, | 0.23% | | |
| | ATP-Sensitive | | | |
| 53 | Potassium Channel, | 2.91% | | |
| | Ca2+ Act., VI | | | |
| 54 | Potassium Channel, | −7.86% | | |
| | Ca2+ Act., VS. | | | |
| 55 | Purinergic, P2Y | 3.61% | | |
| 56 | Serotonin Transporter | 18.69% | | |
| 57 | Serotonin, 5HT1A | 63.94% | 47.40% | |
| 58 | Serotonin, 5HT1D | 2.60% | | |
| 59 | Serotonin, 5HT2A | 11.57% | | |
| 60 | Serotonin, 5HT2C | 20.50% | | |
| 61 | Serotonin, 5HT3 | −3.32% | | |
| 62 | Serotonin, 5HT4 | 0.32% | | |
| 63 | Serotonin, 5HT5A | −1.14% | | |
| 64 | Serotonin, 5HT6 | 36.43% | | |
| 65 | Serotonin, 5HT7 | 26.75% | | |
| 66 | Sigma 1 | 17.94% | | |
| 67 | Sigma 2 | −0.92% | | |
| 68 | Sodium Channel, | 0.14% | | |
| | Site 1 | | | |
| 69 | Sodium Channel, | 9.74% | | |
| | Site 2 | | | |
| 70 | Thromboxane, TXA2 | 25.68% | | |
| 71 | VIP, PACAP SV1 | 15.84% | | |
| 81 | Protease, Caspase 2 | −5.09% | | |
| 82 | Protease, Caspase 3 | 9.36% | | |
| 83 | Acetylcholinesterase | 2.47% | | |
| 84 | Angiotensin II, AT1 | −7.99% | | |
| 85 | Endothelin, ET-A | −1.51% | | |
| 86 | Histamine, H2 | −12.35% | | |
| 87 | Kinase, Tyrosine, | 22.02% | | |
| | p60c-src | | | |
| 88 | Kinase, Tyrosine, | −2.18% | | |
| | b-Insulin Receptor | | | |
| | bIRK) | | | |
| 89 | NOS (Neuronal- | −18.87% | | |
| | Binding) | | | |
| 90 | Protein Phosphatase, | −6.60% | | |
| | PP1 | | | |
| 91 | Protein Phosphatase, | 29.68% | | |
| | PP2C | | | |
| 92 | Protein Tyrosine | 19.26% | | |
| | Phosphatase, PTP1B | | | |
| 93 | Cytochrome P450, | 83.34% | | |
| | CYP1A2 | | | |
| 94 | Cytochrome P450, | −14.15% | | |
| | CYP2A6 | | | |
| 95 | Cytochrome P450, | 86.77% | | |
| | CYP2C19 | | | |
| 96 | Cytochrome P450, | 27.57% | | |
| | CYP2C9*1 | | | |
| 97 | Cytochrome P450, | 27.57% | | |
| | CYP2D6 | | | |
| 98 | Cytochrome P450, | 34.04% | | |
| | CYP3A4 | | | |

### Additional Drawings

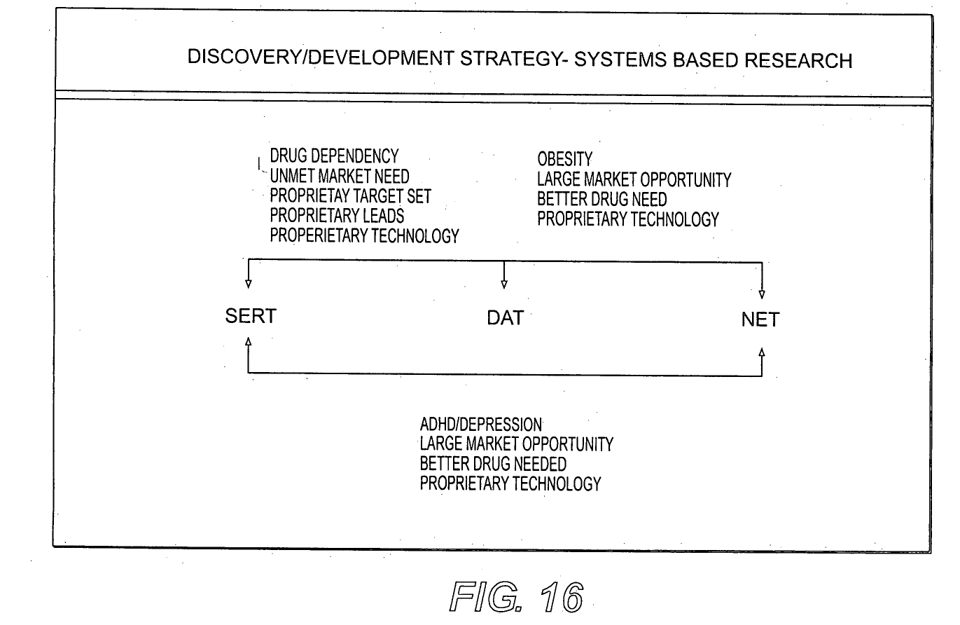

FIG. 16 is a diagram showing three possible molecular targets (DAT=dopamine transporter; SERT=serotonin transporter; NET=norepinephrine transporter) and selected diseases or medical conditions that could potentially be treated with compounds showing differential activity against different target combinations. Specifically, compounds with activity against DAT and SERT, but little or no activity against NET, are potential drugs for treating cocaine addiction. Compounds with activity against DAT and NET, but little or no activity against SERT, are potential drugs for treating obesity. Compounds with activity against NET and SERT, but little or no activity against DAT, are potential drugs for treating depression or attention deficit hyperactivity disorder. Methods disclosed in this invention may be used to identify compounds with positive activity against the two respective targets and relative inactivity against the third target, either by direct interrogation of a database containing results of tests of interactions between a multiplicity of chemical compounds and a multiplicity of molecular targets, or by converting information in such a database to descriptor sets that can be used for in silico screening to identify new compounds with the desired spectrum of activity and relative lack of activity against the selected targets or target combinations.

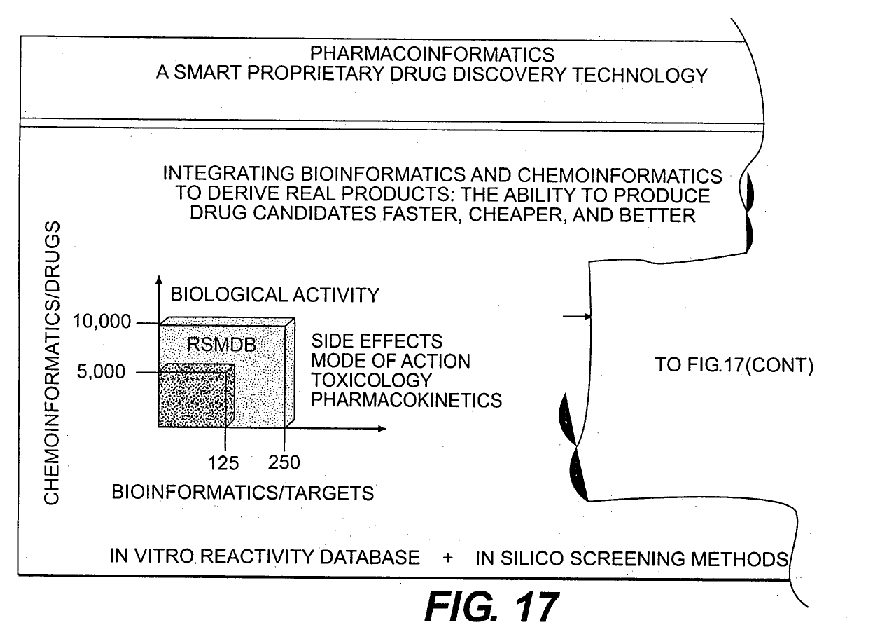

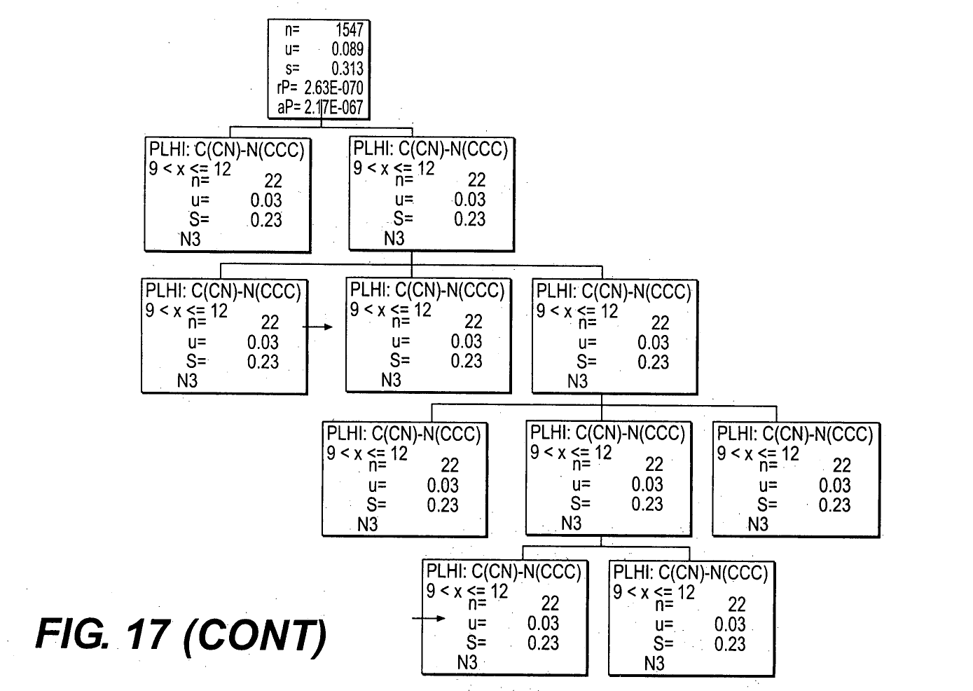

FIG. 17 shows the interrelationship between a pharmacoinformatics database, such as one containing results of tests of interactions between a multiplicity of chemical compounds and a multiplicity of molecular targets, and in silico screening methods, such as use of recursive partitioning to identify descriptor sets associated with measurements or patterns of interactions, pharmacological activity, biological activity, or molecular recognition between descriptor-encoded chemicals and selected Umolecular targets. In addition to mechanism or mode of action or therapeutic effect, targets and information in the database address potential side effects, toxicology, and pharmacokinetic parameters.

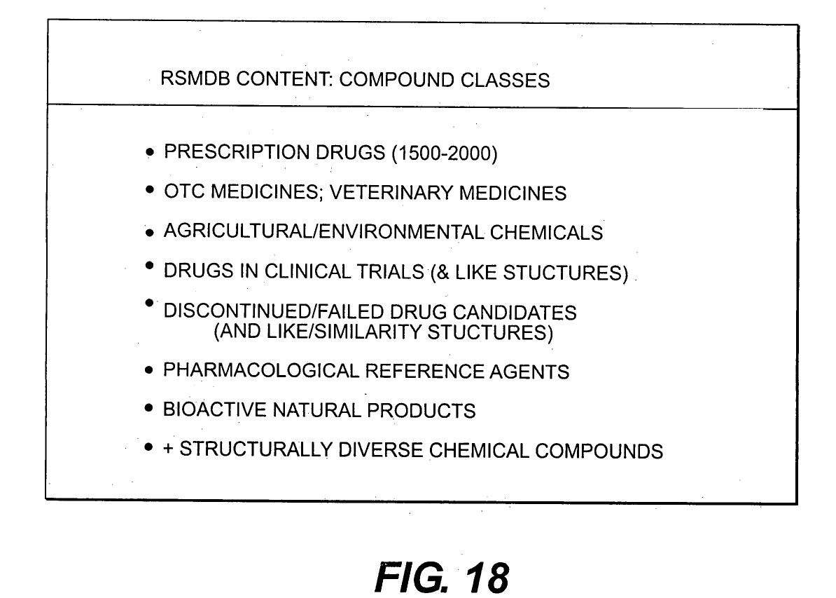

FIG. 18 shows a list of chemical compound types useful for inclusion in a pharmacoinformatics database in the present invention, including different categories of compounds with known biological activity and structurally diverse chemical compounds or diverse compound libraries, the latter of which are particularly useful for identifying new chemical structural features, or pharmacophores, for drug discovery using methods disclosed in this invention.

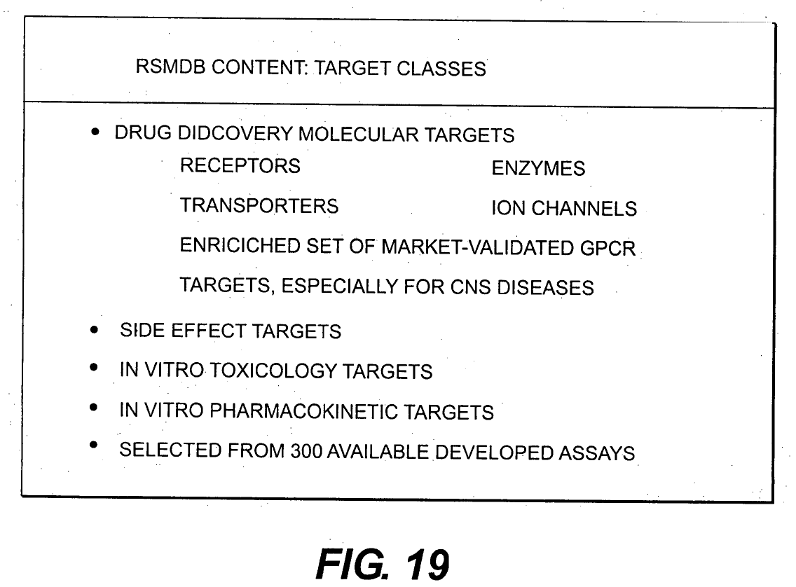

FIG. 19 shows a list of molecular target types useful for inclusion in a pharmacoinformatics database in the present invention, especially including targets relevant to diseases, disease processes, or medical condition associated with the central nervous system, such as psychiatric disorders, neurodegenerative diseases, pain, anxiety, depression, addiction, etc.

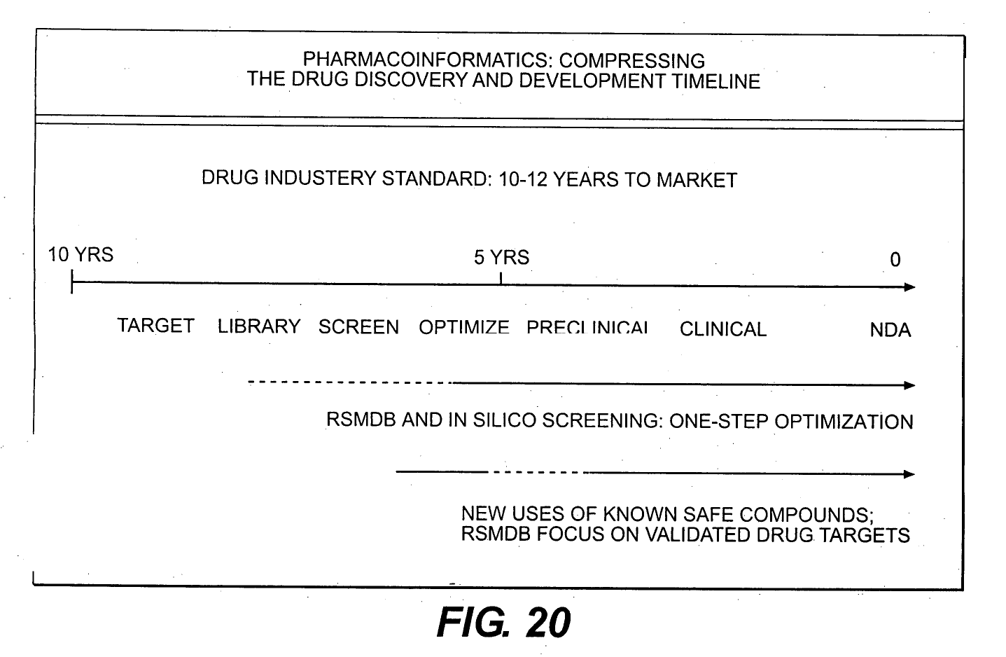

FIG. 20 provides an exemplary timeline showing extensive length of time required for drug discovery and development using current methods and the potential to significantly compress the discovery timeline, thus saving time and money for the pharmaceutical industry, using methods disclosed in this invention, particularly parallel or one-step, instead of sequential, processes for lead compound optimization.

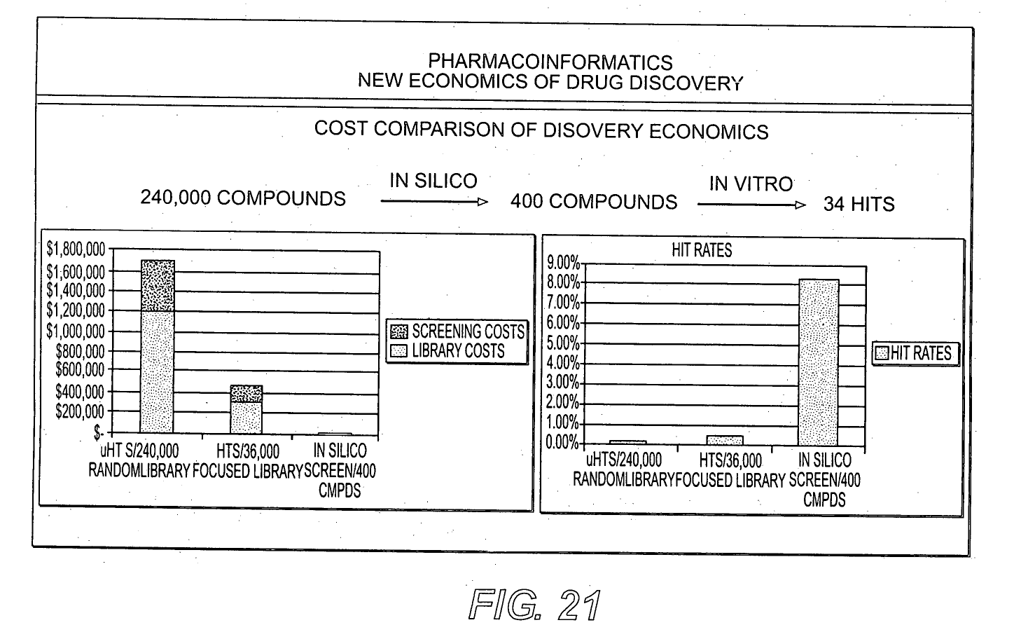

FIG. 21 shows an example of potential time and cost savings by use of methods described in this invention using in silico screening methods to reduce cost of compound library purchases and reduce cost and time for confirmatory in vitro screening of compound sets.

While the present invention has been described in connection with various embodiments, many modifications will be readily apparent to those skilled in the art. One skilled in the art will also appreciate that all or part of the systems and methods consistent with the present invention may be stored on or read from computer-readable media, such as secondary storage devices, like hard disks, floppy disks, and CD-ROM; a carrier wave received from a network such as the Internet; or other forms of ROM or RAM. Accordingly, embodiments of the invention are not limited to the above described embodiments and examples, but instead is defined by the appended claims in light of their full scope of equivalents.

What is claimed is:

1. A method of drug discovery and development comprising using one or more databases comprising chemical and biological interaction data and one or more computer-based data analysis programs to identify compounds that have desired activity at two or more molecular targets that are associated with a disease state for which the drug discovery and development are directed.

2. The method of claim 1, wherein the drug discovery and development are directed to identifying additional applications and uses of known compounds.

3. The method of claim 1, wherein the drug discovery and development are directed to identifying multiple targets relevant to the treatment of a specific disease state.

4. The method of claim 1, wherein the drug discovery and development are directed to in silico identification of compounds that display patterns of activity at two or more molecular targets that are associated with a disease state.

5. A method of drug discovery and development comprising using one or more databases comprising chemical and biological interaction data and one or more computer-based data analysis programs to identify compounds that (a) have desired activity at one or more molecular targets that are associated with a disease state for which the drug discovery and development are directed and (b) do not have activity or have substantially reduced activity that is undesired at one or more molecular targets that are associated with possible side effects; toxicity; adverse absorption, distribution, metabolism, or elimination (ADME) properties; or other properties not intended to be manifested by compounds being developed to treat the disease state associated with the drug discovery.

6. The method of claim 5, wherein the drug discovery and development are directed to identifying additional applications and uses of known compounds.

7. The method of claim 5, wherein the drug discovery and development are directed to identifying multiple targets relevant to the treatment of a specific disease state.

8. The method of claim 5, wherein the drug discovery and development efforts are directed to in silico identification of compounds that display patterns of activity and inactivity at two or more molecular targets that are associated with a disease state.

9. A method of drug discovery comprising:
selecting two or more molecular targets related to a cause or mechanism of a disease, disease process or medical condition;
accessing a dataset comprising results of tests of interactions between each of the selected targets and a multiplicity of chemical compounds, wherein the chemical compounds may be described by descriptors related to features of the compounds;
establishing criteria for selecting, and then selecting a set of active compounds comprising those chemical compounds that demonstrate activity in the tests of interactions between the targets and compounds, for each of the selected molecular targets;
assembling sets of descriptors identified with those compounds comprising the set of selected active compounds, for each of the selected molecular targets;
identifying from the sets of assembled descriptors for each selected molecular target those descriptors that are found in common for each combination of two or more of the selected molecular targets; and
identifying, using the identified in common descriptors, chemical compounds useful for drug discovery purposes related to a disease, disease process, or medical conditions to which the selected molecular targets are related.

10. The method of claim 9, further comprising:
using the identified in common descriptors to access a set of chemical compounds suitable for drug discovery, such compounds being encoded by descriptors that include a form of descriptors used for the method of claim 9;
searching the set of compounds suitable for drug discovery for the presence of the identified in common descriptors and selecting those chemical compounds from the set of compounds suitable for drug discovery that have the features represented by the identified in common descriptors; and
obtaining such selected chemical compounds for use in drug discovery screening processes directed toward a disease, disease process, or medical condition or for other drug discovery purposes.

11. The method of claim 9, wherein the identified in common descriptors are used for design or synthesis of new compounds.

12. The method of claim 11, wherein the design or synthesis of new compounds is directed toward drug discovery related to a disease, disease process, or medical conditions to which the selected molecular targets are related.

13. The method of claim 9, wherein the molecular targets are receptors, enzymes, transporters, uptake sites, ion channels, proteins, nucleic acids, carbohydrates, or polysaccharides.

14. The method of claim 9, wherein the disease, disease process, or medical condition is cocaine addiction, attention deficit hypersensitivity disorder, Parkinson's disease, anxiety, depression, obesity, or barbiturate abuse.

15. The method of claim 9, wherein the dataset of interactions is from a receptor selectivity mapping database.

16. The method of claim 9, wherein the interactions are measured by binding, interaction between a compound known to interact with a target and the target, functional activation, functional enhancement, functional inhibition, or lack of function effect with respect to a molecular target.

17. The method of claim 9, wherein descriptor types are 2-dimensional distance geometries, 3-dimensional distance geometries, sub-structural components, molecular volumes, charge distributions, cnarge distributions, atom types, or descriptors derived by means of physicochemical depiction of a small molecule.

18. The method of claim 9, wherein the selection criteria are dependent on desired or undesired properties of the selected molecular targets.

19. A method for identifying or designing a chemical compound that has desired characteristics and interacts with one or more selected molecular targets, comprising:
selecting one or more positive molecular targets for which a positive interaction with a chemical compound is desired and one or more negative molecular targets for which a lack of significant interaction with the same chemical compound is desired;
accessing a dataset comprising results of tests of interactions between each of the selected positive and negative targets and a multiplicity of chemical compounds, wherein the chemical compounds may be described by descriptors related to features of the compounds;
establishing a threshold or other criteria for selecting, and then selecting a set of active compounds comprising those chemical compounds that demonstrate a desired positive interaction or activity in the tests of interactions between the targets and compounds, for each of the selected positive targets;
establishing a threshold or other criteria for selecting, and then selecting a second set of active compounds comprising those chemical compounds that demonstrate an undesired positive interaction or activity in the tests of interactions between the targets and compounds, for each of the selected negative targets;
assembling sets of positive descriptors that are identified with the compounds comprising the set of selected active compounds for the positive targets;
assembling sets of negative descriptors that are identified with the compounds comprising the second set of selected active compounds for the negative targets; and
using the positive descriptors and negative descriptors to identify or design chemical compounds having characteristics indicative of the positive descriptors but lacking characteristics indicative of the negative descriptors.

20. The method of claim 19, fdLther comprising wherein the positive descriptors and negative descriptors are further used for identifying a chemical compound that has desired characteristics by:
using the positive descriptors and negative descriptors to identify a chemical compound having desired characteristics by accessing a set of chemical compounds potentially suitable for an intended purpose or use, the set of potentially suitable chemical compounds being encoded by descriptors that include a form of descriptors used for the method of claim 12;
searching the set of potentially suitable chemical compounds for the presence of the positive descriptors and selecting a subset of chemical compounds with characteristics indicated by the positive descriptors;
searching the subset of chemical compounds for the presence of the negative descriptors and eliminating chemical compounds from the subset of chemical compounds that have characteristics indicated by the negative descriptors; and
obtaining and further using or testing the eliminated chemical compounds for the intended purpose.

21. The method of claim 20, further comprising obtaining and further using or testing compounds remaining in the subset after having eliminated the chemical compounds that have characteristics indicated by the negative descriptors.

22. The method of claim 20, wherein the set of chemical compounds comprises synthetic chemicals, small organic molecules, natural products, virtual compounds, a virtual library, or drug-like compounds.

23. The method of claim 19, further comprising:
using the positive descriptors and negative descriptors to identify a chemical compound having desired characteristics by accessing a set of chemical compounds potentially suitable for an intended purpose or use, the set of potentially suitable chemical compounds being encoded by descriptors that include a form of descriptors used for the method of claim 12;
searching the set of potentially suitable chemical compounds for the presence of the negative descriptors and eliminating chemical compounds that have characteristics indicated by the negative descriptors;
searching the set of potentially suitable chemical compounds corresponding to remaining compounds for the presence of the positive descriptors and selecting chemical compounds from the remaining compound set that have characteristics indicated by the positive descriptors; and
obtaining and further using or testing the selected remaining compounds for the intended purpose.

24. The method of claim 19, wherein the positive and negative descriptors are used for design of chemical compounds having characteristics indicated by the positive and negative descriptors.

25. The method of claim 24, wherein the designed compounds are synthesized and further used or tested for the intended purpose.

26. The method of claim 19, wherein the method is used for drug discovery or development.

27. The method of claim 19, wherein the positive targets are related to the cause or mechanism of a disease, disease process or medical condition.

28. The method of claim 19, wherein the positive targets are receptors, enzymes, transporters, uptake sites, ion channels, proteins, nucleic acids, carbohydrates, macromolecules, or polysaccharides.

29. The method of claim 19, wherein the negative targets are related to the cause or mechanism of drug side effects, drug adverse effects, toxicity effects, toxicological effects, undesired pharmacokinetic properties, or undesirable effects of administration of pharmaceuticals.

30. The method of claim 19, wherein the negaive targets are receptors, enzymes, transporters, uptake sites, ion channels, proteins, nucleic acids, carbohydrates, macromolecules, or polysaccharides.

31. The method of claim 19, wherein the dataset comprises results of tests of interactions between each of the selected targets and a multiplicity of chemical compounds in a full-rank dataset.

32. The method of claim 19, wherein the dataset comprises results of tests of interactions between each of the selected targets and a multiplicity of chemical compounds that contains positive and negative interaction test results.

33. The method of claim 19, wherein each compound in the dataset has been tested against each target in the dataset, results of each such test being recorded in a database including the dataset.

34. The method of claim 19, wherein the tests of interactions measures an effect that each compound has on an interaction of a compound known to interact with a specific molecular target from the selected positive and negative targets and the specific molecular target.

35. The method of claim 34, wherein the tests of interaction comprise a competitive binding assay.

36. The method of claim 19, wherein the tests of interactions comprise a binding assay.

37. The method of claim 19, wherein the tests of interactions comprise a functional assay.
